(19) United States
(12) Patent Application Publication
(10) Pub. No.: US 2020/0140529 A1
(43) Pub. Date: May 7, 2020

(54) METHODS AND COMPOSITIONS FOR INHIBITING HIV TRANSMISSION

(71) Applicant: REEF PHARMACEUTICALS PTY LTD, VICTORIA (AU)

(72) Inventor: Damian Francis John Purcell, North Balwyn (AU)

(21) Appl. No.: 16/671,742

(22) Filed: Nov. 1, 2019

Related U.S. Application Data
(63) Continuation of application No. 15/818,472, filed on Nov. 20, 2017, now abandoned.
Application No. 15/818,472 is a continuation-in-part of application No. 13/639,831, filed on Oct. 5, 2012, now abandoned.
(60) Provisional application No. 61/322,399, filed on Apr. 9, 2010.

(51) Int. Cl. C07K 16/10

(52) U.S. Cl. CPC C07K 16/1063; A61K 2039/54

(57) ABSTRACT

The present invention provides methods and compositions useful in the field of medicine, and particularly in the treatment of viral infections. More particularly, the invention relates to the use of methods and compositions for the inhibition of human immunodeficiency virus (HIV) transmission.

### CROSS-REFERENCE TO RELATED APPLICATIONS

This application is a Continuation Application of U.S. patent application Ser. No. 15/818,472, filed Nov. 20, 2017, which is a Continuation in Part of U.S. Ser. No. 13/639,831, filed Oct. 5, 2012, which is a Section 371 National Stage Application of International Application No. PCT/AU2011/000407, filed Apr. 11, 2011, published as WO 2011/123900 A1 on Oct. 13, 2011, in English, which is based on and claims the benefit of U.S. Provisional Patent Application No. 61/322,399, filed Apr. 9, 2010; the contents of which are hereby incorporated by reference in their entireties.

### FIELD OF THE INVENTION

The present invention provides methods and compositions useful in the field of medicine, and particularly in the treatment of viral infections. More particularly, the invention relates to the use of methods and compositions for the inhibition of human immunodeficiency virus (HIV) transmission.

### BACKGROUND TO THE INVENTION

The retrovirus designated human immunodeficiency virus (HIV) is the etiological agent of the complex disease that includes progressive destruction of the immune system (acquired immune deficiency syndrome; AIDS) and degeneration of the central and peripheral nervous system. Since it emerged as a public health threat in the early 1980's, efforts to control or eradicate the disease have focused principally on options for treating the disease after an individual has already become infected.

The use of condoms provides a substantial degree of protection against transmission of HIV infections during sexual intercourse. However, the use of condoms is not 100% effective against the transmission of HIV. Moreover, couples often do not use condoms. A topical composition that could be inserted into the vagina or rectum by a foam, gel, sponge or other form, or which could be topically applied to the male genitalia, would in many cases be preferred over condoms. Moreover, the prophylactic effectiveness of condoms could be improved by including a suitable microbicide in the lubricant coated on the exterior of the condom. However, to date little progress has been made to develop an effective topical composition against the transmission of HIV.

Most work to develop topical HIV prophylactic compositions has focused on the use of surfactants and buffers, such as the over-the-counter product nonoxynol-9. Surfactants and detergents disrupt microbial and sperm membranes by lysis and emulsification. Surfactant-containing creams and gels have the advantage of being very broad in their killing ability, and thus can kill the HIV virus and viruses associated with other sexually transmitted diseases.

The use of surfactants and buffers is, however, substantially limited by the damage they can cause to cell membranes. In the vagina, nonoxynol-9 has been shown to thin vaginal walls. In the rectum, nonoxynol-9 can cause rectum walls to slough off.

Other virusidal compositions being investigated for use as HIV virusides include carageenan and other large sulfated polysaccharides that stick to viral envelopes and possibly shield cell membranes. Non-nucleoside inhibitors of the human immunodeficiency virus reverse transcriptase have also been shown to have some effect against HIV.

Scientists have recently reported several biological discoveries that improve our understanding of how HIV enters the host organism following sexual contact, which could lead to prophylactic substances that interfere with HIV's interaction with its target cells.

These discoveries revolve generally around T lymphocytes, monocytes/macrophages and dendritic cells, suggesting that CD4 cell receptors are engaged in the process of virus transmission. For example, it is thought that HIV tightly binds the surface of dendritic cells, and when the dendritic cells present microbial antigens to CD4+ T helper cells to stimulate an immune response, the dendritic cell inadvertently transfers the HIV to the CD4+ T cells, thereby advancing the progression of the infection.

Some have postulated, based upon these discoveries, that prophylactics can be designed that block the interaction between the virus and the human host. However, methods that rely on the specific interaction of HIV and human cells are limited, because the infection pathway has not been fully defined and may be diverse. (Miller, C. J. et al., “Genital Mucosal Transmission of Simian Immunodeficiency Virus: Animal Model for Heterosexual Transmission of Human Immunodeficiency Virus”, J. Virol., 63, 4277-4284 (1989); Phillips, D. M. and Bourinbaiar, A. S., “Mechanism of HIV Spread from Lymphocytes to Epithelia”, Virology, 186, 261-273 (1992); Phillips, D. M., Tan, X., Pearce-Pratt, R. and Zacharopoulos, V. R., “An Assay for H IV Infection of Cultured Human Cervix-derived Cells”, J. Virol. Methods, 52, 1-13 (1995); Ho, J. L. et al., “Neutrophils from Human Immunodeficiency Virus (HIV)-SeronegatiVe Donors Induce HIV Replication from HIV-infected Patients Mononuclear Cells and Cell lines”: An In Vitro Model of HIV Transmission Facilitated by Chlamydia Trachomatis., “J. Exp. Med., 181, 1493-1505 (1995); and Braathen, L. R. & Mork, C. in “HIV infection of Skin Langerhans Cells”, In: Skin Langerhans (dendritic) cells in virus infections and AIDS (ed. Becker, Y.) 131-139 (Kluwer Academic Publishers, Boston, (1991)).

Efforts by researchers to develop an HIV vaccine have also not yet been successful. For example, vaccination with inactivated SIV does not protect African Green monkeys against infection with the homologous virus notwithstanding a strong immune response to SIV.

As will be apparent from the foregoing review of the prior art, there remain significant problems to be overcome in the prevention and treatment of HIV and HIV transmission. It is an aspect of the present invention to overcome or ameliorate a problem of the prior art by providing compositions and methods for the inhibition of HIV transmission.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect the present invention provides a composition for inhibiting transmission of HIV comprising polyclonal antibodies or fragments thereof capable of binding to a human immunodeficiency virus (HIV) viral envelope (Env) protein or a fragment thereof. The Env protein or fragment thereof may be any HIV Env protein, however preferably the Env protein is gp140. Without wishing to be limited by theory, it is proposed that the polyclonal antibodies act to bind HIV virions, thereby inhibiting movement of HIV through cells that form the barrier layer on mucosal surfaces. In one embodiment the composition is capable of preventing HIV infection of a cell.

In one embodiment, the Env protein or fragment thereof is a gp140 oligomer. The oligomer may comprise gp140 trimers, dimers and monomers. The Oligomers may be purified from transduced HeLa and 293 cell supernatant, for example by lentil lectin affinity chromatography and gel filtration. The Env protein or fragment thereof may be a HIV clade A, clade B or clade C strain viral envelope (Env) protein or fragment thereof.

In one embodiment, the Env protein or fragment thereof is a stabilized gp140 trimer. In one embodiment, the gp140 trimer is stabilized by way of covalent bond between residues of any two or more of the six subunits (3× gp120; 3× gp41) of the gp140 trimer. The covalent bond may be formed between a residue of gp120 and a residue of gp41. Preferably the covalent bond is an intermolecular disulphide bond formed between gp120 and gp41. (N. Schülke, et al; J. Virol. 76:7760-7776, 2002; the contents of which is herein incorporated by reference).

Preferably, the stabilized gp140 trimer comprises one or more mutations in gp41 and/or gp120 configured to enhance stability of the trimer. Such mutation(s) are preferably made in combination with introduction of intermolecular covalent bonding between subunits as described supra. Preferably, the mutation is a substitution of a residue in the N-terminal heptad repeat region of gp41. In one embodiment, the mutation is an isoleucine-to-proline substitution at position 559 in the N-terminal heptad repeat region of gp41 (Sanders et al; J. Virol. September 2002 vol. 76 no. 17 8875-8889; the contents of which is herein incorporated by reference)

The stabilized gp140 trimer may be fully cleaved, but remain predominantly trimeric under appropriate conditions. It is proposed animals are immunized with SOSIP gp140 formulated as a vaccine (preferably with an adjuvant) while maintaining conditions favouring the maintenance of trimers in a method for producing neutralizing heterologous neutralizing polyclonal antibodies.

Other mutations in the Env protein which may be useful in the context of the present invention include MPER deletion, which is a deletion in the transmembrane domain on Env to improve solubility and inhibit aggregation. Another mutation is sc-gp140, whereby the cleavage site is replaced with Gly-Ser linkers “6R”, which improves cleavage.

In one embodiment, the polyclonal antibodies or fragments thereof are capable of binding to a Env protein from a heterologous clade of HIV or a heterologous strain of HIV.

In one embodiment the composition comprises polyclonal antibodies or fragments thereof raised against Clade B gp140 that are capable of binding to a heterologous Clade A, Clade B or Clade C gp140.

In one embodiment, the polyclonal antibodies or fragments thereof compete with monoclonal antibody Ab b12 binding to gp140.

In one embodiment, the polyclonal antibodies or fragments thereof compete with, or are, are neutralizing antibodies.

The antibody, or fragment thereof, or functional equivalent thereof may be produced by immunization of an animal with a HIV viral envelope (Env) protein or a fragment thereof.

The animal may be immunized with gp140, recombinant gp140 or oligomeric gp140. The recombinant gp140 may not be derived from virion culture. The animal may be immunized with a HIV viral envelope (Env) protein or a fragment thereof and an adjuvant. In one embodiment, the adjuvant is a water in oil emulsion.

The animal may be immunized with Clade B gp140 and antibodies produced that are capable of binding to a heterologous Clade A, Clade B or Clade C gp140.

The animal may be immunized with Clade B gp140 and antibodies produced compete with monoclonal antibody Ab b12 binding to gp140.

The animal may be immunized with Clade B gp140 and antibodies produced that are capable of binding to a heterologous Clade A, Clade B or Clade C gp140, wherein the antibodies produced compete with monoclonal antibody Ab b12 binding to gp140.

In one embodiment, the composition comprises polyclonal antibodies or fragments thereof capable of binding to a human immunodeficiency virus (HIV) viral envelope (Env) protein or a fragment thereof, wherein the polyclonal antibodies or fragments thereof are capable of binding to a Env protein from a heterologous clade of HIV. The Env protein or fragment thereof may be gp140, and may be a gp140 oligomer. In one embodiment, the Env protein or fragment thereof is a stabilized gp140 trimer, which may be stabilized by way of covalent bond between residues of any two or more of the gp140 trimer. In one embodiment, the covalent bond is formed between a residue of gp120 and a residue of gp41, and may be an intermolecular disulphide bond formed between gp120 and gp41. The stabilized gp140 trimer of the composition may comprise one or more mutations in gp41 and/or gp120 configured to enhance stability of the trimer. The mutation may be a substitution of a residue in the N-terminal heptad repeat region of gp41, and for example may be an isoleucine-to-proline substitution at position 559 in the N-terminal heptad repeat region of gp41. In one embodiment, the Env protein or fragment thereof is an SOSIP gp140, such as BG505 SOSIP or a functional equivalent thereof.

The polyclonal antibodies or fragments thereof raised in response to the HIV Env protein or fragment thereof may be obtained from a milk or a colostrum of an animal. The animal may be an ungulate, such as a member of the family Bovidae. In one embodiment, the ungulate is a cow. The polyclonal antibodies or fragments thereof may have a subset of antibodies with HCDR3 regions of at least 25 amino acids long, or at least 50 amino acids long. The polyclonal antibodies or fragments thereof may be at least partially purified or enriched compared with the milk or colostrum from which they are obtained.

The antibody, or fragment thereof, or functional equivalent thereof may be present in or obtained from an avian egg, or present in or obtained from hyperimmune colostrum or hyperimmune milk of an animal. The animal may be a cow.

The composition may be formulated for topical administration, and in certain embodiments the composition is formulated for vaginal or rectal administration. The composition may be formulated as a gel, or formulated as a topical cream, ointment, lotion or foam formulation.

In certain embodiments, the composition may further comprise a pharmaceutically acceptable excipient, a lubricant, or an antiviral agent.

The present invention also provides the use of a composition of the present invention for the manufacture of a medicament for the treatment and/or prevention of HIV transmission.

The present invention also provides a method of preparing a composition for inhibiting transmission of HIV comprising immunizing an animal with a HIV viral envelope (Env) protein or a fragment thereof, and obtaining hyperimmune colostrum from the immunized animal.

The present invention also provides a composition for inhibiting transmission of H IV prepared by the method comprising immunizing an animal with a HIV viral envelope (Env) protein or a fragment thereof, and obtaining hyperimmune milk from the immunized animal.

The present invention also provides a method of inhibiting transmission of HIV comprising: forming hyperimmune colostrum or hyperimmune milk by immunizing cows; and administering the hyperimmune colostrum or hyperimmune milk to a subject, wherein the step of immunizing cows to produce hyperimmune colostrum or hyperimmune milk comprises vaccination with a human immunodeficiency virus (HIV) viral envelope (Env) protein or a fragment thereof. The Env protein or fragment thereof may be any HIV Env protein, however preferably the Env protein is gp140.

Also provided is a method for inhibiting transmission of HIV comprising administering polyclonal antibodies or fragments thereof capable of binding to a human immunodeficiency virus (HIV) viral envelope (Env) protein or a fragment thereof to a subject. The Env protein or fragment thereof may be any HIV Env protein, however preferably the Env protein is gp140.

In one embodiment, the Env protein or fragment thereof is a gp140 oligomer. The oligomer may comprise gp140 trimers, dimers and monomers. The Oligomers may be purified from transduced HeLa and 293 cell supernatant, for example by lentil lectin affinity chromatography and gel filtration. The Env protein or fragment thereof may be a HIV clade A, clade B or clade C strain viral envelope (Env) protein or fragment thereof.

In one embodiment, the polyclonal antibodies or fragments thereof are capable of binding to a Env protein from a heterologous clade of HIV or a heterologous strain of HIV.

The antibody, or fragment thereof, or functional equivalent thereof may be produced by immunization of an animal with a HIV viral envelope (Env) protein or a fragment thereof.

The animal may be immunized with gp140, recombinant gp140 or oligomeric gp140. The recombinant gp140 may not be derived from virion culture. The animal may be immunized with a HIV viral envelope (Env) protein or a fragment thereof and an adjuvant. In one embodiment, the adjuvant is a water in oil emulsion.

The antibody, or fragment thereof, or functional equivalent thereof may be present in or obtained from an avian egg, or present in or obtained from hyperimmune colostrum or hyperimmune milk of an animal. The animal may be a cow.

The composition may be formulated for topical administration, and in certain embodiments the composition is formulated for vaginal or rectal administration. The composition may be formulated as a gel, or formulated as a topical cream, ointment, lotion or foam formulation.

### BRIEF DESCRIPTION OF THE DRAWINGS

FIG. 1 illustrates a gp140 vaccination schedule.

FIG. 2 illustrates a gp140 vaccination schedule.

FIG. 3 demonstrates IgG from serum and colostrum binds to gp140 Env of clade A, B and C.

FIG. 4 demonstrates purified colostrum IgG from non-pregnant cows retains binding to gp140 Env and demonstrates heterologous binding activity.

FIG. 5 demonstrates bovine IgG blocks binding of monoclonal Ab b12 to CD4 binding site of gp140

FIG. 6 demonstrates colostrum from pregnant cows vaccinated with clade A B/C gp140 and non-pregnant cows vaccinated with clade B gp140 have broad heterologous neutralizing activity.

FIG. 7 demonstrates purified colostrum IgG has neutralizing activity.

FIG. 8 is a timeline detailing the cow vaccination schedule, and sample taking.

FIG. 9 is a diagrammatic representation of the quantitative ELISA used to measure the amount of total bovine IgG, including reagents used.

FIG. 10 is a diagrammatic representation of the binding ELISA used to measure the amount of bovine IgG that is able to bind HIV gp140 Env trimers, including reagents used.

FIG. 11 is a diagrammatic representation of the competition ELISA for binding of bovine polyclonal IgG to Env in preference to binding human reference monoclonal antibodies to neutralising epitopes, such as the CD4bs of HIV Env, including reagents used.

FIG. 12 is a graph showing AD8 gp140 Env-specific binding of colostrum IgG samples at 1/100 dilution raised by immunization of cows with the vaccine and listed below that include subtype-B AD8 Env gp140 oligomers, subtype-A KNH1-SOSIP Env gp140 oligomers, subtype-B PSC89 transmission/founder strain Env gp140 oligomers, and subtype-C MW Env gp140 oligomers. Points above the “Threshold” line represents samples with an endpoint titer above 100. Pooled pre-immune serum from the 32 cows present in the study was used as the negative. The positive control is the highest binding colostrum sample from a previous study. All points represent the mean of 2 replicates. Error bars for individual samples were smaller than the graphical representation of the points and not shown. The mean and standard deviation of each vaccination group are shown.

FIG. 13 is a graph showing subtype-B PSC89 gp140 transmission/founder strain Env-specific binding of colostrum IgG samples at 1/100 dilution raised by immunization of cows with the vaccines listed below that include subtype-B AD8 Env gp140 oligomers, subtype-A KNH1-SOSIP Env gp140 oligomers, subtype-B PSC89 transmission/founder strain Env gp140 oligomers, and subtype-C MW Env gp140 oligomers. Points above the “Threshold” line represents samples with an endpoint titer above 100. Pooled pre-immune serum from the 32 cows present in the study was used as the negative. The positive control is the highest binding colostrum sample from a previous study. All points represent the mean of 2 replicates. Error bars for individual samples were smaller than the graphical representation of the points and not shown. The mean and standard deviation of each vaccination group are shown.

FIG. 14 is a graph showing subtype-C MW-specific binding of colostrum IgG samples at 1/100 dilution raised by immunization of cows with the vaccine and listed below that include subtype-B AD8 Env gp140 oligomers, subtype-A KNH1-SOSIP Env gp140 oligomers, subtype-B PSC89 transmission/founder strain Env gp140 oligomers, and subtype-C MW Env gp140 oligomers. Points above the “Threshold” line represents samples with an endpoint titer above 100. Pooled pre-immune serum from the 32 cows present in the study was used as the negative. The positive control is the highest binding colostrum sample from a previous study. All points represent the mean of 2 replicates. Error bars for individual samples were smaller than the graphical representation of the points and not shown. The mean and standard deviation of each vaccination group are shown.

FIG. 15 is a Table showing the initial vaccination and booster vaccination regimen with SOSIP proteins, or the uncleaved gp140 or uncleaved SEKS gp140 controls that were administered to each of 10 cows after the collection of colostrum post-partum for IgG.

FIG. 16 is a graph showing the average serum IgG concentration over the study. IgG concentration measured by ELISA was average for samples in the time-points before any vaccination, before the revaccination study and at the end of the revaccination study. Values were analyzed using one-way ANOVA.

FIG. 17 is a graph showing AD8-specific titer of serum samples before and after booster re-vaccinations with the SOSIP gp140 or control proteins listed in the Table in FIG. 15. Reciprocal serum endpoint as measured by ELISA relative to the average serum IgG concentrations measured before and after booster vaccinations plotted in FIG. 16. Results represent the mean of 2 replicates and error bars (if any) represent standard deviation. The first column per group represents the titer before vaccination, and the 2nd column the post-revaccination titer.

FIG. 18 is a graph showing MW8 gp140 Env-specific titer of serum samples before and after booster re-vaccinations with the SOSIP gp140 or control proteins listed in the Table in FIG. 15. Reciprocal serum endpoint as measured by ELISA relative to the average serum IgG concentrations measured before and after booster vaccinations plotted in FIG. 16. Results represent the mean of 2 replicates and error bars (if any) represent standard deviation. The first column per group represents the titer before vaccination, and the 2nd column the post-revaccination titer.

FIG. 19 is a graph showing BG505 SOS-IP gp140 Env-specific titer of Serum samples before and after booster re-vaccinations with the SOSIP gp140 or control proteins listed in the Table in FIG. 15. Reciprocal serum endpoint as measured by ELISA relative to the average serum IgG concentrations measured before and after booster vaccinations plotted in FIG. 16. Results represent the mean of 2 replicates and error bars (if any) represent standard deviation. The first column per group represents the titer before vaccination, and the 2nd column the post-revaccination titer.

FIG. 20 is a graph showing fold inhibition of b12 binding to CD4bs by serum IgG. Results represent the mean of 3 replicates and error bars (if any) represent standard deviation. The first column per group represents the fold competition of b12-binding before vaccination, and the 2nd column the post-revaccination competition of b12-binding.

### DETAILED DESCRIPTION OF THE INVENTION

The present invention is predicated in part on the finding that highly specific colostrum antibodies binding to the HIV Env protein can be generated by vaccination of pregnant animals. Accordingly, in a first aspect the present invention provides a composition for inhibiting transmission of HIV comprising polyclonal antibodies or fragments thereof capable of binding to a human immunodeficiency virus (HIV) viral envelope (Env) protein or a fragment thereof. The Env protein or fragment thereof may be any HIV Env protein, however preferably the Env protein is gp140. Without wishing to be limited by theory, it is proposed that the polyclonal antibodies act to bind HIV virions, thereby inhibiting movement of HIV through cells that form the barrier layer on mucosal surfaces. In one embodiment the composition is capable of inhibiting or preventing HIV infection of a cell. In another embodiment the composition is capable of inhibiting or preventing HIV movement through epithelial cells, such as those that form the barrier layer on mucosal surfaces. This approach to formulating compositions and method for inhibiting transmission of HIV is distinguished from approaches of the prior art, and is indeed contrary to the general teaching of the prior art prior to the present invention.

The term “inhibiting transmission” as used herein, generally refers to complete inhibition and also partial inhibition of HIV transmission. Complete inhibition indicates that the HIV virus is completely unable to successfully infect and/or replicate and/or further infect other cells.

This can be determined in a number of ways, at the cellular and/or whole organism level, by the skilled practitioner. One such determination is by an inability to obtain infectious HIV from a host cell. Another such determination is by an inability to determine that HIV has entered the host cell. At the whole organism level, standard methods for assaying for HIV infection can be used (e.g., assaying for antibodies to HIV in the individual). Partial inhibition refers to a measurable, statistically significant reduction in the ability of HIV to infect and/or replicate and/or further infect other cells, as compared to an appropriate control which has not been subjected to the therapeutics described herein. One example would be a requirement for higher levels of exposure or longer period of exposure to HIV for successful infection.

The term “capable of binding” as used herein, generally refers to an antibody that binds to a gp140 of a clade of HIV, such as an antibody described herein. Binding to a gp140 of a clade of HIV may be demonstrated as described in the Examples below. In useful embodiments, the antibodies or fragments thereof bind to a gp140 of a strain or clade of HIV the antibodies are raised against, and also bind to a gp140 of a strain or clade of HIV the antibodies are not raised against. In other useful embodiments, the antibodies or fragments thereof bind to a gp140 of the clade of HIV the antibodies are raised against, and also bind to a gp140 of a heterologous clade of HIV.

The term “clade(s)”, as used herein, generally encompasses subtypes or recombinant forms of HIV.

Previous work has indicated there is a need to provide improved compositions that provide protection against HIV and/or inhibition of transmission of HIV. In particular, there is a need to provide compositions that protect against HIV without compromising the integrity of the innate protective surface layer of the vagina or rectum. Work towards this end has focused on active immunity (e.g. vaccines), however when antibodies against HIV are used in compositions against HIV, these antibodies are made using HIV antigen which would difficult to achieve regulatory approval for and use due to a risk of infection and difficulty of manufacture in volume. In contrast to the teachings of the prior art, the present invention is predicated in part on the provision of passive hetero-immunity, wherein antibodies made in a particular organism are used to protect another organism, generally a different species.

The Env ectodomain is known as gp140, which contains both gp120 and truncated gp41 (lacking transmembrane domains and cytoplasmic tails). In one embodiment, the Env protein or fragment thereof is a gp140 oligomer. The oligomer may comprise gp140 trimers, dimers and monomers. The Oligomers may be purified from transduced cell (e.g. HeLa and 293) supernatant, for example by lentil lectin affinity chromatography and gel filtration. The Env protein or fragment thereof may be a HIV clade A, clade B or clade C strain viral envelope (Env) protein or fragment thereof.

In a related application (PCT/AU2009/001218, incorporated herein by reference), Applicants have demonstrated strains of HIV-1 have differences in their Envs.

In one embodiment the composition comprises polyclonal antibodies or fragments thereof capable of binding to a Clade A, Clade B or Clade C gp140.

In another embodiment the composition comprises polyclonal antibodies or fragments thereof capable of binding to a Clade A and a Clade B gp140.

In another embodiment the composition comprises polyclonal antibodies or fragments thereof capable of binding to a Clade B and a Clade C gp140.

In another embodiment the composition comprises polyclonal antibodies or fragments thereof capable of binding to a Clade A and a Clade C gp140.

In another embodiment the composition comprises polyclonal antibodies or fragments thereof capable of binding to a Clade A, a Clade B and a Clade C gp140.

In one embodiment the composition comprises polyclonal antibodies or fragments thereof raised against Clade B gp140 that are capable of binding to a heterologous Clade A, Clade B or Clade C gp140.

In one embodiment, the composition comprises polyclonal antibodies or fragments thereof that compete with monoclonal antibody Ab b12 binding to gp140.

In another embodiment, the composition comprises polyclonal antibodies or fragments thereof raised against Clade B gp140 that are capable of binding to a heterologous Clade A, Clade B or Clade C gp140, wherein the antibodies or fragments thereof compete with monoclonal antibody Ab b12 binding to gp140.

Surprisingly, Applicants have demonstrated colostrum, and IgG purified from colostrum, from cows vaccinated with HIV Env gp140 oligomers of one clade can bind HIV Env gp140 of another clade, despite diversity in Env sequence and glycosylation across HIV strains.

Furthermore, applicants have demonstrated colostrum, and IgG purified from colostrum, from cows vaccinated with HIV Env gp140 oligomers of one clade can bind gp140 and neutralize HIV of another clade, despite diversity in Env sequence and glycosylation across HIV strains.

Dairy cows were vaccinated in the second trimester of pregnancy with high quality soluble oligomeric HIV-1 Env (gp140) to produce colostrum containing high levels of HIV-1 Env-specific polyclonal neutralizing antibodies for use as an HIV transmission inhibiting composition. The present inventors have shown that anti-HIV Env IgG synergizes with intrinsic antiviral components in bovine colostrum to aggressively neutralize HIV-1.

Accordingly, the present invention provides an advantage of the production of kilogram quantities of bovine IgG. Without wishing to be bound by theory, the compositions of the present invention are proposed to have potent ability to neutralise HIV and thereby render it non-infectious for susceptible cells in vitro.

In one embodiment, the polyclonal antibodies or fragments thereof are neutralizing antibodies.

The term “neutralisation” as used herein, generally refers to antibodies or fragments thereof that are able to bind the molecule of the invention and hamper its biological activity. The term encompasses antibodies or fragments thereof that block a virus, e.g. HIV, from infecting a cell by, for example, blocking gp140 binding to CD4 on a cell.

The antibody, or fragment thereof, or functional equivalent thereof may be produced by immunization of an animal with a HIV viral envelope (Env) protein or a fragment thereof.

The animal may be immunized with gp140, recombinant gp140 or oligomeric gp140.

The animal may be immunized with a clade B gp140 and the antibodies produced are capable of binding to a heterologous Clade A, Clade B or Clade C gp140.

The animal may be immunized with Clade B gp140 and antibodies produced that are capable of binding to a heterologous Clade A, Clade B or Clade C gp140.

The animal may be immunized with Clade B gp140 and antibodies produced compete with monoclonal antibody Ab b12 binding to gp140.

The animal may be immunized with Clade B gp140 and antibodies produced that are capable of binding to a heterologous Clade A, Clade B or Clade C gp140, wherein the antibodies produced compete with monoclonal antibody Ab b12 binding to gp140.

In one embodiment, the polyclonal antibodies or fragments thereof produced are neutralizing antibodies.

In one embodiment, the polyclonal antibodies or fragments thereof produced block binding of gp140 to CD4 on a cell.

In one embodiment, the recombinant gp140 may not be derived from virion culture.

In one embodiment, HIV-1 Env covalently stabilized into the compact trimeric form known as SOSIP gp140 is used for the elicitation of polyclonal antibodies capable of neutralizing infection of HIV, and useful in the formulation of a topical microbiocide. Preferably, the SOSIP gp140 is BG505 SOSIP (Sanders, R. W. et al; PLoS Pathog. 9, e1003618 (2013), or KNH SOSIP, or AD8 SOSIP, the contents of which is herein incorporated by reference and with primary data).

An Env polypeptide that is suitable to generate an immune response is an Env polypeptide having at least 90%, 95%, 96%, 97%, 98%, 99% or 100% amino acid identity to gp140. gp140 contains a fragment of a gp120 from a given HIV strain and a fragment of gp41 from the same H IV strain, wherein the soluble gp41 fragment lacks the transmembrane domain. gp140 polypeptides capable of forming oligomeric structures may be expressed from a construct. Accordingly, there is provided a nucleotide sequence that encodes for a gp140 polypeptide, and an expression construct comprising a nucleotide sequence that codes for a gp140 polypeptide. The expression construct may be one for transient use or be more suitable for stable transfection and maintenance within a target cell as either an episomally replicating construct or an integrated form.

In one embodiment there is provided a gp140 polypeptide, and an expression construct that expresses a gp140 polypeptide. In another embodiment, there is provided a glycosylated gp140 polypeptide, which has been manufactured or expressed in an expression system that adds the native cellular glycosylation.

Recombinant gp140 may be produced using pseudoviruses carrying Env from different clades/strains using the expression vectors included in Table 1.gp140 may be purified by a number of different means. For example, gp140-containing tissue culture supernatants may be passed over lentil lectin affinity columns, which mediate the capturing of glycoproteins, including gp140, through the affinity of lentil lectin for carbohydrate. After washing, gp140 is eluted competitively from the column by the addition of 0.5M Methyl-D-mannopyranoside (Sigma). Yields obtained with this system for other gp140 strains have varied between 0.4 and 1.0 milligram per 100 millilitres of tissue culture supernatant. The eluate may then be concentrated and further purified by gel filtration over superdex 200. The gp140 is purified from the culture supernatants in an oligomeric form.

Soluble Env gp140 oligomers have been prepared from clade A, B, and C HIV-1 strains from HeLa and/or 293T cells and purified by lentil lectin affinity and gel filtration chromatography.

Four cows (two pregnant in second semester and two initially non-pregnant) were vaccinated with 100 g of purified HIV-1 Env gp140 oligomer formulated with Montanide adjuvant. Two groups of two cows (one pregnant and one nonpregnant) were vaccinated with either clade B (AD8) only or with equal amounts (33.33 pg) of clade A, B and C Env gp140 (UG8, AD8 and MW) (referred to as ‘trimix’). All four cows received at least three vaccinations whereas the last vaccination was given four weeks before giving birth. All four cows seroconverted within nine weeks. Reciprocal endpoint serum IgG titers were up to 1×10^{2 5} for pregnant cows and up to 1×10⁵ for non-pregnant cows determined by a new established anti-bovine IgG HIV-1 Env gp140 specific ELISA. The expected low serum IgG titer in pregnant cows was explained by the pumping of serum IgG antibodies into the colostrum approximately four weeks before giving birth.

HIV-immune bovine colostrum was collected and pasteurised postpartum from all cows with pregnancy vaccination resulting in relatively low responses with reciprocal IgG titers of <10² (clade B vaccinated) and 1×10³ s (trimix-vaccinated). Reciprocal colostrum IgG titer for cows vaccinated before pregnancy was 10⁵ (clade B vaccinated) and 10′⁵ (trimix vaccinated). Western blot analysis confirmed that colostrum IgG of all four cows was specific against HIV-1 Env gp140. Unfractionated colostrum was tested for neutralising activity in a HIV-1 Env-pseudotyped reporter virus assay.

In brief, Env-pseudotyped reporter virus assay detects the presence of virus-neutralising antibodies to HIV-1 envelope protein. EGFP reporter pseudovirus particles expressing HIV-1 Env derived from strains/clades of choice are used to infect target cells in an Env dependent manner. In one assay, the reporter pseudovirus particles are incubated for 1 hour before the addition of the target cells (Cf2th-CD4/CCR5/CXCR4; CF2 cells) at 2×10⁴/well in a 96-well plate. After a 2-hour spinoculation at 1200× g at room temperature, residual pseudovirus and antibody was removed and fresh media added to the cells. Two days later the target cells are analysed for EGFP expression by FACS. In the presence of colostrum or colostrum IgG raised against soluble HIV-1 Env gp140 oligomers, the degree of reduction in the level of infection was determined by measuring the reduction in the percent EGFP positive cells. The neutralisation percentage represents a ratio between infection levels observed in mice sera before vaccination (pre-bleed) and mice sera 2 weeks post protein boost 3 vaccination.

Clade A/E, clade B and clade C pseudotype viruses including the NIH reference panel for clade B and C viruses were tested (total n=27) and compared with non-immune bovine colostrum that already has intrinsic infection-blocking activity due to lactoferrin and other bioactive peptides. Unfractionated colostrum from the trimix cow vaccinated during pregnancy showed high neutralisation of up to 50% for all B clade pseudoviruses (n=15) as well as for the majority of C clade (n=11) and clade A/E (n=1) pseudoviruses at a dilution of 1:16. The first clade B vaccinated cow was a low responder but both cows vaccinated before pregnancy and having their calves recently responded well. Up to this time, broad neutralisation was observed for the clade B vaccinated cow that showed 50-80% neutralisation for B clade (n=12) and clade C pseudoviruses (n=9) (1:16 dilution) (Table 1). IgG Abs from the first pair of cows was purified from the colostrum and neutralising activity was retained for purified IgG with up to 50% neutralisation for the trimix-IgG compared to non immune IgG at 500 g/ml. Results of the neutralisation profile of two HIV Env gp140 hyperimmune bovine colostrum samples against pseudoviruses of different clades are demonstrated in Table 1 (see, Example 2). This result is surprising since antibodies raised against gp140 are not expected to bind and neutralize viruses of different clades strongly, since there are significant epitope differences between the gp140 of the different clades.

These results strongly support this method of raising high levels of neutralising antibodies, and neutralizing antibodies that can bind heterologous HIV strains. Accordingly, in one embodiment, the polyclonal antibodies or fragments thereof are capable of binding to an Env protein from a heterologous clade of HIV or a heterologous strain of HIV.

In one embodiment the composition comprises polyclonal antibodies or fragments thereof capable of binding to a Clade A, Clade B or Clade C gp140.

In another embodiment the composition comprises polyclonal antibodies or fragments thereof capable of binding to a Clade A and a Clade B gp140.

In another embodiment the composition comprises polyclonal antibodies or fragments thereof capable of binding to a Clade B and a Clade C gp140.

In another embodiment the composition comprises polyclonal antibodies or fragments thereof capable of binding to a Clade A and a Clade C gp140.

In another embodiment the composition comprises polyclonal antibodies or fragments thereof capable of binding to a Clade A, a Clade B and a Clade C gp140.

In another embodiment the composition comprises polyclonal antibodies or fragments thereof raised against Clade B gp140 that are capable of binding to a heterologous Clade A, Clade B or Clade C gp140.

In another embodiment the composition comprises polyclonal antibodies or fragments thereof that compete with monoclonal antibody Ab b12 binding to gp140.

In another embodiment, the polyclonal antibodies or fragments thereof are neutralizing antibodies.

In another embodiment the composition comprises polyclonal antibodies or fragments thereof raised against Clade B gp140 that are capable of binding to a heterologous Clade A, Clade B or Clade C gp140, wherein the polyclonal antibodies or fragments thereof compete with monoclonal antibody Ab b12 binding to gp140.

The antibody, or fragment thereof, or functional equivalent thereof may be present in or obtained from an avian egg, or present in or obtained from hyperimmune colostrum or hyperimmune milk of an animal. The animal may be a cow.

Methods for generating hyperimmune sera, milk, colostra and the like are known in the art.

The method for generating the hymperimmune material may comprise the step of purifying the Env protein from other potentially immunogenic molecules. For example, Env proteins can isolated by methods such as high and low speed centrifugation, optionally with the use of gradients formed using sucrose, percoll, cesium and the like. Chromotagraphic methods such as size exclusion chromatography, affinity chromatography, high performance liquid chromatography, reverse phase chromatography, and the like are also useful. Electrophoretic methods (such as capillary electrophoresis), filtration methods (such as tangential flow ultrafiltration), partitioning methods (such as protein precipitation) are further examples of useful methods. Chronically infected cell lines may be developed by infection of cells, e.g. 6D5 cells (a subclone of the HUT78 cell line) with HIV. Radioimmunoprecipitation analysis is used to examine that the cell line secretes Env into the medium. The Env protein may then be purified from the serum-free conditioned medium by affinity chromatography using mouse MAbs to the Env protein.

For the production of hyperimmune material, the Env protein (whether or not purified) is administered to an animal, typically by way of injection (for example, via the IM, subcutanteous, intraperitoneal, or intravenous route). The Env protein may be combined with an adjuvant to increase the immune response generated by the animal.

Accordingly, the animal may be immunized with a HIV viral envelope (Env) protein or a fragment thereof and an adjuvant. In one embodiment, the adjuvant is a water in oil emulsion.

The skilled person is familiar with many potentially useful adjuvants, such as Freund's complete adjuvant, alum, and squalene. Adjuvants which may be used in compositions of the invention include, but are not limited to oil emulsion compositions suitable for use as adjuvants in the invention include oil-in-water emulsions and water-in-oil emulsions, complete Freund's adjuvant (CFA) and incomplete Freund's adjuvant (IFA) may also be used. Preferably, Montanide brand adjuvants may be used (e.g. MONTANIDE ISA 50V, MONTANIDE ISA 206, and MONTANIDE IMS 1312). These adjuvants are oily adjuvant compositions of mannide oleate and mineral oil, or water based nanoparticles combined with a soluble immunostimulant.

Adjuvants suitable for use in the invention include bacterial or microbial derivatives such as derivatives of enterobacterial lipopolysaccharide (LPS), Lipid A derivatives, immunostiinulatory oligonucleotides and ADP-ribosylating toxins and detoxified derivatives thereof.

The animal may be dosed with Env at intervals over a period of days, weeks or months. At the conclusion of the immunization regime, the hyperimmune material (such as blood, milk or colostrums) is harvested. Antibodies in the hyperimmune material may be harvested by any suitable method, including any by method described supra.

In one embodiment the composition comprises antibodies from colostrum or a colostrum extract, further characterised in that the colostrum is enriched in anti-Env antibodies when compared with colostrum obtained without vaccination.

In one embodiment of the method the polyclonal antibodies are obtained from a hyperimmune material. The hyperimmune material is enriched when compared with corresponding material in which the animal has not been challenged with the antigen in question.

The animal used to produce the hyperimmune material may be any suitable animal, including a human. However, since human milk may contain potentially transmissible human pathogens, one form of the method provides that the antibody is not human-derived. In any event, animals that produce large quantities of milk are preferred. In this regard, ungulates (and cows in particular), are animals useful for the generation of hyperimmune material.

The use of ungulates (and particularly cows) is proposed to provide advantage in so far as the antibodies produced by these animals are able to access an occluded conserved epitopes on HIV Env, such as the CD4-binding site defined with b12 and VRC01 monoclonal antibodies. The surface epitopes of Env are relatively variable across clades, and antibodies directed to those epitopes are therefore limited in their ability to neutralize a virus of a heterologous clade.

The ungulate is preferably an even-toed ungulate (Order Artiodactyla), more preferably a ruminant (Ruminantia), more preferably horned livestock (Cervoidea; Pecora), more preferably a bovid (Bovidae).

BG505 SOSIP gp140 has been used to immunize rabbits (McCoy, L. E. et al; Cell Rep 16, 2327-2338 (2016)). and primates (Sanders, R. W. et al. Science 349, aac4223-aac4223 (2015), although in both animals failed to produce broadly neutralizing antibodies. This is in contrast to the present invention, which has discovered that ungulates are a useful source of broadly neutralizing antibodies.

Without wishing to be limited by theory in any way, it is proposed that the ability of ungulate antibodies to access occluded conserved epitopes of gp140 relates to the presence of a relatively long third heavy chain complementarity determining region (HCDR3). A subset of ungulate polyclonal antibodies is known to have HCDR3 over 70 amino acids in length de los Rios, M., et al, Curr. Opin. Struct. Biol. 33, 27-41 (2015); Wang, F. et al. Reshaping antibody diversity. Cell 153, 1379-1393 (2013)).

Thus, in some embodiments of the invention, the HIV env-specific polyclonal antibodies comprise a subset of antibodies having a HCDR3 comprising at least about 10, 15, 20, 25, 30, 35, 40, 45, 50, 55, 60, 65, 70, 75, 80, 85, 90 or amino acids. Preferably the HIV env-specific polyclonal antibodies comprise a subset of antibodies having a HCDR3 comprising at least about 25 amino acids.

The subset of antibodies may comprise at least about 1, 2, 3, 4, 5, 6, 7, 8, 9, 10, 11, 12, 13, 14, 15, 16, 17, 18, 19, 20, 25, 30, 35, 40 or 45% of all antibodies in the population.

It will be understood that antibodies of some non-ungulate species may have a similarly relatively long HCDR3, and it is proposed that such non-ungulate species will be useful sources of broadly neutralizing antibodies according to the present invention.

In one embodiment of the method, the “hyperimmune material” is hyperimmune dairy derived material such as milk particularly colostral milk (colostrum) and the like which is enriched in antibodies or fragments thereof and which is derived from an animal source. The hyperimmune dairy material is preferably hyperimmune colostrum.

In another embodiment the hyperimmune material is derived from bird eggs. A subtype of immunoglobulin known as IgY can be easily extracted from the yolk. Typically, the yolk is first defatted and the IgY isolated by methods identical or similar to those used for skim milk.

The term “colostrum” as used herein includes colostral milk; processed colostral milk such as colostral milk processed to partly or completely remove one or more of fat, cellular debris, lactose and casein; and colostral milk or processed colostral milk which has been dried by for example, freeze drying, spray drying or other methods of drying known in the art. Colostral milk is generally taken from a mammal such as a cow within five days after parturition. Preferably the mammalian colostrum is bovine colostrum retained from the first 4 days post parturition, more preferably bovine colostrum

retained from the first 2 days post parturition, even more preferably bovine colostrum retained from the first day post parturition, and most preferably bovine colostrum retained from the first milking post parturition.

Preferably the colostrum collected from the cow comprises at least 4% total protein (weight 10%), more preferably 5%, more preferably at least 8%, more preferably at least 10%.

Preferably the ratio of IgG to total protein of the colostrum collected from the cow is at least 10%, more preferably 20%.

The hyperimmune dairy material preferably contains at least 3 g per kilogram of product which is IgG directed against Env, or an equivalent molar concentration of the anti-Env antibody. For example the hyperimmune material may contain at least 5 g, at least 10 g or at least 15 g anti-Env antibody per kg of hyperimmune material on the basis of the dry weight of components. The upper end of the range of antibody concentration will depend on factors such as the dose, the disease state and the health of the patient. The hyperimmune material may, for example contain no more than 80 g such as no more than 60 g, no more than 50 g or no more than 40 g anti-Env antibody per kg of hyperimmune material on the basis of the dry weight of components.

In one embodiment of the method the polyclonal antibodies are administered to the subject as a composition. The composition may in one embodiment comprise a carrier admixed with the ligand prior to administration, for example, by mixing a composition of hyperimmune colostrum from immunized cows or one or more processed components thereof with conventional foods and/or pharmaceutically acceptable excipients. The ratio of enriched product relative to conventional dairy material from unvaccinated animals may, for example, be at least 4, such as at least 10 in a comparative ELISA assay.

In another embodiment part or all of the antibodies specific for Env are extracted from the colostrum and used to prepare a composition for administration.

In one embodiment the hyperimmune material binds Env derived from a clade A, clade B or clade C HIV-1 strain. Preferably the hyperimmune material binds at least two of the above, more preferably at least 3 of the clades. The degree of enrichment in material selected from antibodies capable of binding to Env may be at least 4 times, for example at least 10 times the level found in corresponding unvaccinated animals with respect each of the Env molecules as determined by standard ELISA.

In one embodiment, low molecular weight moieties have been substantially removed from the colostrum or the colostrum extract. By substantially removed is meant that at least 75% and preferably 90% of the low molecular weight moieties are removed.

In a preferred example of this embodiment at least 75% (such as at least 90% or substantially complete removal) of, moieties of molecular weight less than 30 kDa have been removed from the colostrum or the colostrum extract. Preferably molecular weight moieties less than 60 kDa have been substantially removed from the colostrum or colostrum extract.

In one embodiment, the hyperimmune material comprises immunogenic material selected from antibody and antibody fragments which bind Env. Preferably the antibody or antibody fragment is a polyclonal antibody or a polyclonal antibody fragment of bovine origin.

The composition may further contain growth factor molecules that are normally found in milk or colostrum. These factors may produce a synergism with the anti-Env antibodies contained in the composition. Exemplary growth factors include TGF-beta-1, TGF-beta-2, IGF-1, IGF-2, EGF, FGF and PDGF.

The composition may be formulated for topical administration, and in certain embodiments the composition is formulated for vaginal or rectal administration. The composition may be formulated as a gel, or formulated as a topical cream, ointment, lotion or foam formulation.

The topical formulations of the present invention can be used to prevent HIV infection in a human, or to inhibit transmission of the HIV virus from an infected human to another human. The topical formulations of the present invention can inhibit the growth or replication of a virus, such as a retrovirus, in particular a human immunodeficiency virus, specifically HIV-1 and HIV-2. The topical formulations are useful in the prophylactic treatment of humans who are at risk for viral infection. The topical formulations also can be used to treat objects or materials, such as contraceptive devices (for example condoms or intrauterine devices), medical equipment, supplies, or fluids, including biological fluids, such as blood, blood products, and tissues, to prevent or inhibit viral infection of a human. Such topical formulations also are useful to prevent transmission, such as sexual transmission of viral infections, e.g., HIV, which is the primary way in which HIV is transmitted globally. The methods of prevention or inhibition or retardation of transmission of viral infection, e.g., HIV infection, in accordance with the present invention, comprise vaginal, rectal, penile or other topical treatment with an antiviral effective amount of a topical preparation of the present invention, alone or in combination with another antiviral compound as described herein.

Preferred compositions can take several forms. Thus, in one embodiment the composition is in the form of a cream, lotion, gel, or foam that is applied to the affected skin or epithelial cavity, and preferably spread over the entire skin or epithelial surface which is at risk of contact with bodily fluids. Such formulations, which are suitable for vaginal or rectal administration, may be present as aqueous or oily suspensions, solutions or emulsions (liquid formulations) containing in addition to the active ingredient, such carriers as are known in the art to be appropriate. For “stand-alone” lubricants (i.e., lubricants that are not pre-packaged with condoms), gels and similar aqueous formulations are generally preferred, for various reasons (both scientific and economic) known to those skilled in the art. These formulations are useful to protect not only against sexual transmission of HIV, but also to prevent infection of a baby during passage through the birth canal. Thus the vaginal administration can take place prior to sexual intercourse, during sexual intercourse, and immediately prior to childbirth.

One method of applying an anti-viral lubricant to the genitals, for the purposes disclosed herein, involves removing a small quantity (such as a teaspoon, or several millilitres) of a gel, cream, ointment, emulsion, or similar formulation from a plastic or metallic tube, jar, or similar container, or from a sealed plastic, metallic or other packet containing a single dose of such composition, and spreading the composition across the surface of the penis immediately before intercourse. Alternate methods of emplacement include: (1) spreading the composition upon accessible surfaces inside the vagina or rectum shortly before intercourse; and (2) emplacing a condom, diaphragm, or similar device, which has already been coated or otherwise contacted with an anti-viral lubricant, upon the penis or inside the vagina. In a preferred embodiment, any of these methods of spreading an anti-viral lubricant across the surfaces of the genitals causes the lubricant to coat and remain in contact with the genital and epithelial surfaces throughout intercourse.

In another embodiment, the present invention involves topical administration of the topical formulation to the anus. The composition administered to the anus is suitably a foam or gel, etc., such as those described above with regard to vaginal application. In the case of anal application, it may be preferred to use an applicator which distributes the composition substantially evenly throughout the anus. For example, a suitable applicator is a tube 2.5 to 25 cm, preferably 5 to 10 cm, in length having holes distributed regularly along its length.

When the composition is a water-soluble vaginal cream or gel, suitably 0.1 to 4 grams, preferably about 0.5 to 2 grams, are applied. When the composition is a vaginal spray-foam, suitably 0.1 to 2 grams, preferably about 0.5 to 1 grams, of the spray-foam are applied. When the composition is an anal cream or gel, suitably 0.1 to 4 grams, preferably about 0.5 to 2 grams of the cream or gel is applied. When the composition is an anal spray-foam, suitably 0.1 to 2 grams, preferably about 0.5 to 1 grams of the spray-foam are applied.

As a vaginal formulation, the active ingredient may be used in conjunction with a spermicide and may be employed with a condom, diaphragm, sponge or other contraceptive device. Examples of suitable spermicides include nonylphenoxypolyoxyethylene glycol (nonoxynol 9), benzethonium chloride, and chlorindanol. Suitably, the pH of the composition is 4.5 to 8.5. Vaginal compositions preferably have a pH of 4.5 to 6, most preferably about 5.

Vaginal formulations also include suppositories (for example, gel-covered creams), tablets and films. The suppositories can be administered by insertion with an applicator using methods well known in the art.

Typical buccal formulations are creams, ointments, gels, tablets or films that comprise ingredients that are safe when administered via the mouth cavity. Buccal formulations can also comprise a taste-masking or flavoring agent.

The present compositions may also be in the form of a time-release composition. In this embodiment, the composition is incorporated in a composition which will release the active compound at a rate which will result in the vaginal or anal concentration described above. Time-release compositions are disclosed in Controlled Release of Pesticides and Pharmaceuticals, D. H. Lew, Ed., Plenum Press, New York, 1981; and U.S. Pat. Nos. 5,185,155; 5,248,700; 4,011,312; 3,887,699; 5,143,731; 3,640,741; 4,895,724; 4,795,642; Bodmeier et al, Journal of Pharmaceutical Sciences, vol. 78 (1989); Amies, Journal of Pathology and Bacteriology, vol. 77 (1959); and Pfister et al, Journal of Controlled Release, vol. 3, pp. 229-233 (1986), all of which are incorporated herein by reference.

The present compositions may also be in the form which releases the composition in response to some event such as vaginal or anal intercourse. For example, the composition may contain the anti-Env antibodies in vesicles or liposomes which are disrupted by the mechanical action of intercourse. Compositions comprising liposomes are described in U.S. Pat. No. 5,231,112 and Deamer and Uster, “Liposome Preparation: Methods and Mechanisms”, in Liposomes, pp. 27-51 (1983); Sessa et al, J. Biol. Chem., vol. 245, pp. 3295-3300 (1970); Journal of Pharmaceutics and Pharmacology, vol. 34, pp. 473-474 (1982); and Topics in Pharmaceutical Sciences, D. D. Breimer and P. Speiser, Eds., Elsevier, New York, pp. 345-358 (1985), which are incorporated herein by reference.

It should also be realized that the present compositions may be associated with a contraceptive device or article, such as a vaginal ring device, an intrauterine device (IUD), vaginal diaphragm, vaginal sponge, pessary, condom, etc. In the case of an IUD or diaphragm, time-release and/or mechanical-release compositions may be preferred, while in the case of condoms, mechanical-release compositions are preferred.

A suitable vaginal ring drug delivery system for slow release of the anti-Env antibodies is disclosed in U.S. Pat. No. 5,989,581, incorporated herein by reference. As described in U.S. Pat. No. 5,989,581, the vaginal ring delivers two actives for contraception. The drug delivery system disclosed comprises at least one compartment comprising a drug dissolved in a thermoplastic polymer core and a thermoplastic skin covering the core. Preferred thermoplastic polymers for both the core and the skin are ethylene-vinylacetate copolymers. As would be understood by one skilled in the art, according to the present invention, the disclosed delivery system contains at anti-Env antibodies useful to prevent, inhibit or slow infection or transmission of HIV. In certain embodiments, said vaginal ring device may also contain one or more additional drugs, for instance a contraceptive agent such as a steroidal progestogenic compound and/or a steroidal estrogenic compound. In yet other embodiments, the vaginal ring system containing a anti-Env antibodies may also contain or be used in combination with a topical estriol, such as Ovestin™, to enhance prevention of infection or transmission of HIV through the vaginal epithelium.

In another embodiment, the present invention provides novel articles which are useful for the prevention or retardation of HIV infection. In particular, the present articles are those which release anti-Env antibodies when placed on an appropriate body part or in an appropriate body cavity. Thus, the present article may be a vaginal ring device as described above or an ILID. Suitable ILIDs are disclosed in U.S. Pat. Nos. 3,888,975 and 4,283,325 which are incorporated herein by reference.

The present article may be an intravaginal sponge which comprises and releases, in a time-controlled fashion, the anti-Env antibodies. Intravaginal sponges are disclosed in U.S. Pat. Nos. 3,916,898 and 4,360,013, which are incorporated herein by reference. The present article may also be a vaginal dispenser which releases the anti-Env antibodies. Vaginal dispensers are disclosed in U.S. Pat. No. 4,961,931, which is incorporated herein by reference.

In one embodiment the compositions are used in conjunction with condoms, to enhance the risk-reducing effectiveness of condoms and provide maximum protection for users. The composition can either be coated onto condoms during manufacture, and enclosed within conventional watertight plastic or foil packages that contain one condom per package, or it can be manually applied by a user to either the inside or the outside of a condom, immediately before use.

As used herein, “condom” refers to a barrier device which is used to provide a watertight physical barrier between male and female genitalia during sexual intercourse, and which is removed after intercourse. This term includes conventional condoms that cover the penis; it also includes so-called “female condoms” which are inserted into the vaginal cavity prior to intercourse. The term “condom” does not include diaphragms, cervical caps or other barrier devices that cover only a portion of the epithelial membranes inside the vaginal cavity. Preferably, condoms should be made of latex or a synthetic plastic material such as polyurethane, since these provide a high degree of protection against viruses.

In another embodiment the compositions are used in conjunction with other possible surfaces for transmission, such as gloves, to provide maximum protection for users. The composition can either be coated onto gloves during manufacture, and enclosed within conventional watertight plastic or foil packages that contain one pair of gloved per package, or it can be manually applied by a user to either the inside or the outside of a glove, immediately before use.

In another embodiment the composition is in the form of an intra-vaginal pill, an intra-rectal pill, or a suppository. The suppository or pill should be inserted into the vaginal or rectal cavity in a manner that permits the suppository or pill, as it dissolves or erodes, to coat the vaginal or rectal walls with a prophylactic layer of the anti-HIV agent.

In still another embodiment the composition is topically applied by release from an intravaginal device. Devices such as vaginal rings, vaginal sponges, diaphragms, cervical caps, female condoms, and the like can be readily adapted to release the composition into the vaginal cavity after insertion.

In certain embodiments, the composition may further comprise a pharmaceutically acceptable excipient, a lubricant, or an antiviral agent.

Compositions used in the methods of this invention may also comprise other active agents, such as another agent to prevent HIV infection, and agents that protect individuals from conception and other sexually transmitted diseases. Thus, in another embodiment the compositions used in this invention further comprise a second anti-HIV agent, a virucide effective against viral infections other than HIV, and/or a spermicide.

In one particular embodiment, the composition contains nonoxynol, a widely-used spermicidal surfactant. The resulting composition could be regarded as a “bi-functional” composition, since it would have two active agents that provide two different desired functions, in a relatively inert carrier liquid; the nonoxynol would provide a spermicidal contraceptive agent, and the polyclonal antibodies or fragments thereof would provide anti-viral properties. The nonoxynol is likely to cause some level of irritation, in at least some users; this is a regrettable but is a well-known side effect of spermicidal surfactants such as nonoxynol and octoxynol, which attack and destroy the lipid bilayer membranes that surround sperm cells and other mammalian cells.

The compositions used in this invention may also contain a lubricant that facilitates application of the composition to the desired areas of skin and epithelial tissue, and reduces friction during sexual intercourse. In the case of a pill or suppository, the lubricant can be applied to the exterior of the dosage form to facilitate insertion.

In still another embodiment the invention provides a device for inhibiting the sexual transmission of HIV comprising (a) a barrier structure for insertion into the vaginal cavity, and (b) a composition comprising a polyclonal antibody according to the present invention. As mentioned above, preferred devices which act as barrier structures, and which can be adapted to apply anti-HIV agent, include the vaginal sponge, diaphragm, cervical cap, or condom (male or female).

In the cream or ointment embodiments of the present invention, the topical formulation comprises one or more lubricants. The gels and foams of the present invention optionally can include one or more lubricants.

Non-limiting examples of useful lubricants include cetyl esters wax, hydrogenated vegetable oil, magnesium stearate, methyl stearate, mineral oil, polyoxyethylene-polyoxypropylene copolymer, polyethylene glycol, polyvinyl alcohol, sodium lauryl sulfate, white wax, or mixtures of two or more of the above.

The amount of lubricant in the topical formulation can range from about 0 to about 95 weight percent. Typical cream and ointment formulations comprise 0.1 to 95 weight percent of lubricant.

The topical formulations can comprise one or more adjuvants, wherein the adjuvant is an antimicrobial agent, antioxidant, humectant or emulsifier, or mixture of two or more thereof. The gels and foams of the present invention can include one or more antimicrobial agents and optionally can include one or more of antioxidants, humectants and emulsifiers.

Non-limiting examples of useful antimicrobial agents are benzyl alcohol, propylene glycol, propyl paraben, methyl paraben, or mixtures of two or more thereof.

The amount of antimicrobial agents in the topical formulation can range from about 0.01 to about 10 weight percent, and in some embodiments from about 0.2 to about 10 weight percent, on a basis of total weight of the topical formulation.

Non-limiting examples of useful antioxidants include butylated hydroxyanisole, butylated hydroxytoluene, edetate disodium or mixtures of two or more thereof.

The amount of antioxidant in the topical formulation can range from about 0.01 to about 1 weight percent, and in some embodiments from about 0.01 to about 0.1 weight percent, on a basis of total weight of the topical formulation.

Non-limiting examples of useful humectants include ethylene glycol, glycerin, sorbitol or mixtures of two or more thereof.

The amount of humectant in the topical formulation can range from about 1 to about 30 weight percent, and in some embodiments from about 2 to about 20 weight percent, on a basis of total weight of the topical formulation.

Non-limiting examples of useful emulsifiers include acrylic acid polymers (such as carbomer brand thickeners e.g. Carbomer 934P, manufactured by Voveon, inc.), polyoxyethylene-10-stearyl ether, polyoxyethylene-20-stearyl ether, cetostearyl alcohol, cetyl alcohol, cholesterol, diglycol stearate, glyceryl monostearate, glyceryl stearate, polygeyceryl-3-oleate, hydroxypropyl cellulose, hydroxypropylmethyl cellulose, lanolin, polyoxyethylene lauryl ether, methyl cellulose, polyoxyethylene stearate, polysorbate, propylene glycol monostearate, sorbitan esters, stearic acid or mixtures of two or more thereof.

The amount of emulsifier in the topical formulation can range from about 1 to about 40 weight percent, and in some embodiments from about 5 to about 30 weight percent, on a basis of total weight of the topical formulation.

The gel formulations of the present invention comprise one or more gelling agents. Non-limiting examples of useful gelling agents include carboxylic acid polymers including acrylic acid polymers crosslinked with cross links such as allyl ethers of sucrose (e.g. carbomer brand thickeners), cetostearyl alcohol, hydroxymethyl cellulose, polyoxyethylene-polyoxypropylene copolymer, sodium carboxymethylcellulose, polyvinyl pyrrolidone, or mixtures of two or more thereof.

The amount of gelling agent in the topical gel formulation can range from about 0.1 to about 10 weight percent, and in some embodiments from about 0.1 to about 1 weight percent, on a basis of total weight of the topical formulation.

The gel formulations of the present invention can further comprise one or more alkalinizers, for example sodium hydroxide, in amount of less than about 2 weight percent as activators of gelling.

The formulations can contain one or more additional excipients well known in the art, for example water and a thickening agent such as colloidal silicon dioxide.

The formulations of the present invention can be administered in combination with one or more other antiviral or other agents useful in treating or preventing infection with HIV or in inhibiting transmission of HIV, in combination with a pharmaceutically acceptable carrier. In one form of the method, the subject is under treatment with an antiretroviral agent.

In some embodiments, the method comprises co-administration of an antiretroviral agent, and particularly an agent used for the treatment of HIV infection such as Zidovudine (AZT), Abacavir, Emtricitabine (FTC), Lamivudine (3TC), Didanosine (ddl), Stavudine (d4T), Zalcitabine (ddC), Nevirapine, Efavirenz, Delavirdine, Tenofovir, Enfuvirtide (T20), Maraviroc (CCR5), Lopinavir, Atazanavir, Fosamprenvir, Amprenavir, Saquinavir, Indinavir, Nelfinavir, Raltegravir, and Elvitegravir.

One or more, preferably one to four, antiviral agents useful in anti-H IV-1 therapy may be used in combination with at least one (i.e., 1-4, preferably 1) anti-Env antibody in a formulation of the present invention. The antiviral agent or agents may be combined with the anti-Env antibody in a single dosage form, or the anti-Env antibody and the antiviral agent or agents may be administered simultaneously or sequentially as separate dosage forms. For example, the anti-Env antibody formulation can be used in a vaginal ring device or to coat the outside of a condom to prevent transmission of HIV to a non-infected sexual partner while the HIV-infected sexual partner undergoes treatment with systemic antiviral therapy. The antiviral agents contemplated for use in combination with the anti-Env antibody formulations of the present invention comprise nucleoside and nucleotide reverse transcriptase inhibitors, non-nucleoside reverse transcriptase inhibitors, protease inhibitors and other antiviral drugs listed below not falling within these classifications. In particular, the combinations known as HAART are contemplated for use in combination with the anti-Env antibody formulations of this invention. [00169] The term “nucleoside and nucleotide reverse transcriptase inhibitors” (“NRTI” s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA.

Typical suitable NRTIs include zidovudine (AZT) available under the RETROVIR tradename from Glaxo-Wellcome Inc., Research Triangle, NC 27709; didanosine (ddl) available under the VIDEX tradename from Bristol-Myers Squibb Co., Princeton, N.J. 08543; zalcitabine (ddC) available under the HMD tradename from Roche Pharmaceuticals, Nutley, N.J. 071 10; stavudine

(d4T) available under the ZERIT trademark from Bristol-Myers Squibb Co., Princeton, N.J. 08543; lamivudine (3TC) available under the EPIVIR tradename from Glaxo-Smith Kline Triangle, NC 27709; abacavir (1592U89) disclosed in WO96/30025 and available under the ZIAGEN trademark from Glaxo-Wellcome Research Triangle, NC 27709; adefovir dipivoxil [bis(POM)-PMEA] available under the PREVON tradename from Gilead Sciences, Foster City, Calif. 94404; lobucavir (BMS-180194), a nucleoside reverse transcriptase inhibitor disclosed in EP-0358154 and EP-0736533 and under development by Bristol-Myers Squibb, Princeton, N.J. 08543; BCH-10652, a reverse transcriptase inhibitor (in the form of a racemic mixture of BCH-10618 and BCH-10619) under development by Biochem Pharma, Laval, Quebec H7V, 4A7, Canada; emitricitabine [(−)-FTC] licensed from Emory University under Emory Univ. U.S. Pat. No. 5,814,639 and available from Gilead under the trade name Emtrivia™; beta-L-FD4 (also called beta-L-D4C and named beta-L-2′, 3′-dicleoxy-5-fluoro-cytidene) licensed by Yale University to Vion Pharmaceuticals, New Haven Conn. 0651 1; DAPD, the purine nucleoside, (−)-beta-D-2,6-diamino-purine dioxolane disclosed in EP 0656778 and licensed by Emory University and the University of Georgia to Triangle Pharmaceuticals, Durham, N.C. 27707; and lodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc., West Conshohoken, Pa. 19428. [00171] The term “non-nucleoside reverse transcriptase inhibitors” (“NNRTII 1 S) as used herein means non-nucleosides that inhibit the activity of HIV-1 reverse transcriptase.

Typical suitable NNRTIs include nevirapine (BI-RG-587) available under the VIRAMUNE tradename from Boehringer Ingelheim, the manufacturer for Roxane Laboratories, Columbus, Ohio 43216; delaviradine (BHAP, U-90152) available under the RESCRIPTOR tradename from Pharmacia & Upjohn Co., Bridgewater N.J. 08807; efavirenz (DMP-266) a benzoxazin-2-one disclosed in WO94/03440 and available under the SUSTIVA tradename from Bristol Myers Squibb in the US and Merck in Europe; PNU-142721, a furopyridine-thio-pyrimide under development by Pharmacia and Upjohn, Bridgewater N.J. 08807; AG-1549 (formerly Shionogi # S-1 153); 5-(3,5-dichlorophenyl)-thio-4-isopropyl-1-(4-pyridyl)methyl-IH-imidazol-2-ylmethyl carbonate disclosed in WO 96/10019 and under clinical development by Agouron Pharmaceuticals, Inc., LaJolla CA 92037-1020; MKC-442 (1-(ethoxy-methyl)-5-(1-methylethyl)-6-(phenylmethyl)-(2,4(1H,3H)-pyrimidinedione) discovered by Mitsubishi Chemical Co. and under development by Triangle Pharmaceuticals, Durham, N.C. 27707; (+)-calanolide A (NSC-675451) and B, coumarin derivatives disclosed in NIH U.S. Pat. No. 5,489,697, licensed to Med Chem Research, which is co-developing (+) calanolide A with Vita-Invest as an orally administrable product; and etravirine (TMC-125, Intelence) marketed by Tibotec. [00173] The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character, e.g. CRIXIVAN (available from Merck) as well as nonpeptide protease inhibitors e.g., VIRACEPT (available from Agouron).

Typical suitable Pis include saquinavir (Ro 31-8959) available in hard gel capsules under the INVIRASE tradename and as soft gel capsules under the FORTOVASE tradename from Roche Pharmaceuticals, Nutley, N.J. 071 10-1 199; ritonavir (ABT-538) available under the NORVIR tradename from Abbott Laboratories, Abbott Park, Ill. 60064; indinavir (MK-639) available under the CRIXIVAN tradename from Merck & Co., Inc., West Point, Pa. 19486-0004; nelfnavir (AG-1343) available under the VIRACEPT tradename from Agouron Pharmaceuticals, Inc., LaJolla CA 92037-1020; amprenavir (141 W94), tradename AGENERASE, a non-peptide protease inhibitor under development by Vertex Pharmaceuticals, Inc., Cambridge, Mass. 02139-421 1 and available from Glaxo-Wellcome, Research Triangle, NC under an expanded access program; lasinavir (BMS-234475) available from Bristol-Myers Squibb, Princeton, N.J. 08543 (originally discovered by Novartis, Basel, Switzerland (CGP-61755); DMP-450, a cyclic urea discovered by Dupont and under development by Triangle Pharmaceuticals; BMS-2322623, an azapeptide under development by Bristol-Myers Squibb, Princeton, N.J. 08543, as a 2nd-generation HIV-1 PI; ABT-378 under development by Abbott, Abbott Park, Ill. 60064; AG-1549 an orally active imidazole carbamate discovered by Shionogi (Shionogi # S-1 153) and under development by Agouron Pharmaceuticals, Inc., LaJolla CA 92037-1020; atazanavir; tipranavir; and darunavir.

Other antiviral agents include CXCR4 antagonists, enfuvirtide, hydroxyurea, ribavirin, IL-2, IL-12, pentafuside and Yissum Project No. 1 1607. Hydroxyurea (Droxia), a ribonucleoside triphosphate reductase inhibitor, the enzyme involved in the activation of T-cells, was discovered at the NCI and is under development by Bristol-Myers Squibb; in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. RE 33653, 4530787, 4569790, 4604377, 4748234, 4752585, and 4949314, and is available under the PROLEUKIN (aldesleukin) tradename from Chiron Corp., Emeryville, Calif. 94608-2997 as a lyophilized powder for IV infusion or sc administration upon reconstitution and dilution with water, a dose of about 1 to about 20 million ILJ/day, sc is preferred; a dose of about 15 million 1 U/day, sc is more preferred. IL-12 is disclosed in WO96/25171 and is available from Roche Pharmaceuticals, Nutley, N.J. 071 10-1 199 and American Home Products, Madison, N.J. 07940; a dose of about 0.5 microgram/kg/day to about 10 microgram/kg/day, sc is preferred. Enfuvirtide (DP-178, T-20) a 36-amino acid synthetic peptide, is disclosed in U.S. Pat. No. 5,464,933 licensed from Duke University to Trimeris which developed enfuvirtide in collaboration with Duke University and Roche; enfuvirtide acts by inhibiting fusion of HIV-1 to target membranes. Enfuvirtide (3-100 mg/day) is given as a continuous sc infusion or injection together with efavirenz and 2 Pi's to HIV-1 positive patients refractory to a triple combination therapy; use of 100 mg/day is preferred. Yissum Project No. 1 1607, a synthetic protein based on the HIV-1 Vif protein, is under preclinical development by Yissum Research Development Co., Jerusalem 91042, Israel. Ribavirin, I-β-D-ribofuranosyl-1H-1,2,4-triazole-3-carboxamide, is available from ICN Pharmaceuticals, Inc., Costa Mesa, Calif.; its manufacture and formulation are described in U.S. Pat. No. 4,211,771; the integrase inhibitor raltegravir available from Merck under the tradename Isentress™; elvitegravir an intergrase inhibitor under development by Gilead Sciences; the H IV-1 Gag maturation inhibitor berivimat under development (Phase lib) by Panacos Pharmaceuticals. [00176] The term “anti-HIV-1 therapy” as used herein means any anti-H IV-1 drug found useful for treating H IV-1 infections in man alone, or as part of multidrug combination therapies, especially the HAART triple and quadruple combination therapies. Typical suitable known anti-HIV-1 therapies include, but are not limited to multidrug combination therapies such as (i) at least three anti-HIV-1 drugs selected from two NRTIs, one PI, a second PI, and one NNRTI; and (ii) at least two anti-HIV-1 drugs selected from NNRTIs and Pis. Typical suitable HAART-multidrug combination therapies include: [00177] (a) triple combination therapies such as two NRTIs and one PI; or (b) two NRTIs and one NNRTI; and (c) quadruple combination therapies such as two NRTIs, one PI and a second PI or one NNRTI. In treatment of naive patients, it is preferred to start anti-HIV-1 treatment with the triple combination therapy; the use of two NRTIs and one NNRTI or two NRTIs and one PI is preferred if there is intolerance to NNRTI. Drug compliance is essential. The CD4+ and HIV-1-RNA plasma levels should be monitored every 3-6 months. Should viral load plateau, a fourth drug, e.g., one PI, one NNRTI or integrase inhibitor could be added.

The present invention also provides the use of a composition of the present invention for the manufacture of a medicament for the treatment and/or prevention of HIV transmission.

The present invention also provides a method of preparing a composition for inhibiting transmission of HIV comprising immunizing an animal with a HIV viral envelope (Env) protein or a fragment thereof, and obtaining hyperimmune colostrum from the immunized animal.

The present invention also provides a composition for inhibiting transmission of H IV prepared by the method comprising immunizing an animal with a HIV viral envelope (Env) protein or a fragment thereof, and obtaining hyperimmune milk from the immunized animal.

The present invention also provides a method of inhibiting transmission of HIV comprising: forming hyperimmune colostrum or hyperimmune milk by immunizing cows; and administering the hyperimmune colostrum or hyperimmune milk to a subject, wherein the step of immunizing cows to produce hyperimmune colostrum or hyperimmune milk comprises vaccination with a human immunodeficiency virus (HIV) viral envelope (Env) protein or a fragment thereof. The Env protein or fragment thereof may be any HIV Env protein, however preferably the Env protein is gp140.

Also provided is a method for inhibiting transmission of HIV comprising administering polyclonal antibodies or fragments thereof capable of binding to a human immunodeficiency virus (HIV) viral envelope (Env) protein or a fragment thereof to a subject. The Env protein or fragment thereof may be any HIV Env protein, however preferably the Env protein is gp140.

In one embodiment, the Env protein or fragment thereof is a gp140 oligomer. The oligomer may comprise gp140 trimers, dimers and monomers. The Oligomers may be purified from transduced HeLa and 293 cell supernatant, for example by lentil lectin affinity chromatography and gel filtration. The Env protein or fragment thereof may be a HIV clade A, clade B or clade C strain viral envelope (Env) protein or fragment thereof.

In one embodiment, the polyclonal antibodies or fragments thereof are capable of binding to a Env protein from a heterologous clade of HIV or a heterologous strain of HIV.

The antibody, or fragment thereof, or functional equivalent thereof may be produced by immunization of an animal with a HIV viral envelope (Env) protein or a fragment thereof. The animal may be immunized with gp140, recombinant gp140 or oligomeric gp140. The recombinant gp140 may not be derived from virion culture. The animal may be immunized with a HIV viral envelope (Env) protein or a fragment thereof and an adjuvant. In one embodiment, the adjuvant is a water in oil emulsion.

The antibody, or fragment thereof, or functional equivalent thereof may be present in or obtained from an avian egg, or present in or obtained from hyperimmune colostrum or hyperimmune milk of an animal. The animal may be a cow.

The composition may be formulated for topical administration, and in certain embodiments the composition is formulated for vaginal or rectal administration. The composition may be formulated as a gel, or formulated as a topical cream, ointment, lotion or foam formulation.

In one embodiment of the method, the ligand is an antibody, or fragment or derivative thereof. The antibodies or fragment or derivative thereof may be polyclonal immunoglobulins or chimeric antibodies or dendrimer presented immunoactive fragments or immunoactive fragments such as F(ab) and F(ab)2 fragments or recombinant immunoactive fragments, or affinity purified immunoglobulins or immunoactive fragments thereof.

In one form of the composition the antibody or fragment thereof or derivative thereof is produced by immunization of an animal with a microbe or a microbial product. Polyclonal antibodies capable of binding to a microbe or microbial product may be obtained by the immunization of an animal, and obtaining the antibodies via a bodily fluid, such as blood, a secretion of a gland or cell, egg, milk or colostrum.

The methods, compositions and devices of this invention can be adapted generally to release active agent in a time sensitive manner that best corresponds to the timing of sexual activity. When topically applied as a lotion or gel, the compositions are preferably applied immediately prior to sexual activity. Other modes of application, such as devices and suppositories, can be designed to release active agent over a prolonged period of time, at a predetermined rate, depending upon the needs of the consumer.

In certain embodiments of the present invention, the goal of the formulations of the present invention is to reduce the HIV-1-RNA viral load below the detectable limit so that infection or transmission of infection is slowed, prevented or inhibited. The “detectable limit of HIV-1-RNA” in the context of the present invention means that there are fewer than about 200 to fewer than about 50 copies of HIV-1-RNA per ml of plasma of the patient as measured by quantitative, multi-cycle reverse transcriptase PCR methodology. HIV-1-RNA is preferably measured in the present invention by the methodology of Amplicor-1 Monitor 1.5 (available from Roche Diagnostics) or of Nuclisens HIV-1 QT-1.

In certain embodiments, the formulations of the invention are useful to protect not only against sexual transmission of HIV, but also to prevent infection of a baby during passage through the birth canal. Thus the vaginal administration can take place prior to sexual intercourse, during sexual intercourse, immediately prior to childbirth or during childbirth. Such topical dosage forms may be particularly useful when applied to a newborn baby of an HIV-infected mother.

The present invention will now be more fully described by reference to the following non-limiting Examples.

### EXAMPLES

### Example 1: Production of Hyperimmune Colostrum Containing Polyclonal Anti-Env Antibodies

Step 1—Production of vaccine for dairy cattle

The procedures for preparing antigen reported in Pub. No. WO/2004/078209 International Application No. PCT/AU2004/000277 (the contents of which are herein incorporated by reference) were used.

Step 2—The procedures for preparing antibodies from vaccinated cattle reported in Pub. No. WO/2004/078209 International Application No. PCT/AU2004/000277 (the contents of which are herein incorporated by reference) were used.

### Example 2: Production of Polyclonal Antibodies Binding to HIV Env, and Demonstration of Neutralization

Soluble Env gp140 oligomers have been prepared from clade A, B, and C HIV-1 strains from HeLa and/or 293T cells and purified by lentil lectin affinity and gel filtration chromatography.

Four cows (two pregnant in second semester and two initially non-pregnant) were vaccinated with 100 g of purified HIV-1 Env gp140 oligomer formulated with Montanide adjuvant. Two groups of two cows (one pregnant and one nonpregnant) were vaccinated with either clade B (AD8) only or with equal amounts (33.33 Mg) of clade A, B and C Env gp140 (UG8, AD8 and MW) (referred to as ‘trimix’). All four cows received at least three vaccinations whereas the last vaccination was given four weeks before giving birth. All four cows seroconverted within nine weeks. Reciprocal endpoint serum IgG titers were up to 1×10² s for pregnant cows and up to 1×10⁵ for non-pregnant cows determined by a new established anti-bovine IgG HIV-1 Env gp140 specific ELISA. The expected low serum IgG titer in pregnant cows was explained by the pumping of serum IgG antibodies into the colostrum approximately four weeks before giving birth.

HIV-immune bovine colostrum was collected and pasteurised postpartum from all cows with pregnancy vaccination resulting in relatively low responses with reciprocal IgG titers of <10² (clade B vaccinated) and 1×10³ s (trimix-vaccinated). Reciprocal colostrum IgG titer for cows vaccinated before pregnancy was 10⁵ (clade B vaccinated) and 10′⁵ (trimix vaccinated). Western blot analysis confirmed that colostrum IgG of all four cows was specific against HIV-1 Env gp140. Unfractionated colostrum was tested for neutralising activity in a HIV-1 Env-pseudotyped reporter virus assay. Clade A E, clade B and clade C pseudotype viruses including the NIH reference panel for clade B and C viruses were tested (total n=27) and compared with non-immune bovine colostrum that already has intrinsic infection-blocking activity due to lactoferrin and other bioactive peptides. Unfractionated colostrum from the trimix cow vaccinated during pregnancy showed high neutralisation of up to 50% for all B clade pseudoviruses (n=15) as well as for the majority of C clade (n=1 1) and clade A E (n=1) pseudoviruses at a dilution of 1:16. The first clade B vaccinated cow was a low responder but both cows vaccinated before pregnancy and having their calves recently responded well. Up to this time, broad neutralisation was observed for the clade B vaccinated cow that showed 50-80% neutralisation for B clade (n=12) and clade C pseudoviruses (n=9) (1:16 dilution) (Table 1). IgG Abs from the first pair of cows was purified from the colostrum and neutralising activity was retained for purified IgG with up to 50% neutralisation for the trimix-IgG compared to non immune IgG at 500 g/ml.

Results of the neutralisation profile of two HIV Env gp140 hyperimmune bovine colostrum samples against pseudoviruses of different clades are demonstrated in Table 1.

These results strongly support this method of raising high levels of neutralising antibodies.

TABLE 1

| | | Neutralisation (%) (1:16) | | |
|---|---|---|---|---|
| | | | | non- |
| Animal number | Clade | 6055 | 7004 | immune |
| Reciprocal IgG titer in H2BC | | 103.5 | 105 | / |
| gp140 specificity | | + | + | / |
| Immunisation/Ag | | Trimix | clase B | / |
| | | AE/B/C | | |
| Pregnant at first vaccination | | + | − | / |
| Env Pseudovirus | | | | |
| AD8 | B | | 74.3 | 64 |
| NL4.3 | B | | 59.1 | 49.7 |
| SF162 | B | | 68.9 | 30.9 |
| 89.6 | B | | | 30.1 |
| MN | B | 54.1 | | 10.2 |
| 11017 6535 clone 3 SVPB5 | B | 63.7 | | 37.3 |
| 11018 QH0692 clone 42 | B | | | 43.4 |
| (SVPB6) | | | | |
| 11022 PVO clone 4 SVPB11 | B | 55.6 | | 25.2 |
| 11023 TRO clone 11, SVPB12 | B | 65.9 | | 34.7 |
| 11024 Ac10.0 clone 29 SVPB13 | B | 63.1 | | 28.4 |
| 11033 pWITO4160 clone33 | B | 53.6 | | 31.2 |
| (SVPB 18) | | | | |
| 11034 pTRJO4551 clone 18 | B | 52.3 | * | 15.5 |
| SVPB 17 | | | | |
| 11035 pREJO4541 clone 67 | B | 63.1 | | 26.3 |
| (SVPB14) | | | | |
| 11036 pRHPA4259 clone 7 | B | 55.4 | * | 13.6 |
| SVPB14 | | | | |
| 11058 SC422661.8 (SVPB8) | B | 61.7 | * | 26.5 |
| 966 | A/E | 30.8 | * | 13.7 |
| 11306 DU 156.12 | C | 43.7 | 42.8 | 28.6 |
| 11307 DU 172.17 | C | 73.8 | 68.9 | 54 |
| 11309 ZM 197M.PB7 | C | 52.3 | 73.1 | 41.8 |
| 11310 ZM 214M.PL15 | C | 50 | 67.7 | 36.2 |
| 11311 ZM 233M.PB6 | C | 61.7 | 77.4 | 30.3 |
| 11312 ZM 249M.PL1 | C | 51 | 83.5 | 36.5 |
| 11316 CAP45.2.00.G3 | C | 33.7 | 57.9 | 37.2 |
| 11317 CAP210.2.00.E8 | C | 55.1 | 87 | 50 |
| 11313 ZM53M.PB12 | C | 50.2 | * | 23.7 |
| 11314 ZM109F.PB4 | C | 47.7 | 81.5 | 22.2 |
| 11315 ZM135M.PL10a | C | 80.1 | * | 30.1 |

Neutralisation profile: green 0%-25%, yellow 25%-50%; orange 50%-75%; red 75%-100%;
* pending

### Example 3: Polyclonal Neutralising Antibodies to HIV-1 Env from Bovine Colostrum

Twelve BSE-free pregnant cows housed in an approved quarantine farm in Victoria are vaccinated with 100 g of an equimolar mix of four HIV-1 Env gp140 oligomers:

1) SC35 clade B pre-seroconversion strain Env gp140, these adopt an open configuration and prominently displays important neutralisation epitopes;

2) ADA primary R5-tropic clade B Env gp140;

3) 966 clade A/E Env gp140; and

4) MW clade C Env gp140.

These Env are formulated with adjuvant (Montanide) and administered twice before pregnancy and at least twice at 3-week intervals during the second trimester of pregnancy by a registered veterinarian.

An Env ELISA assay and Western blotting are used to monitor the levels of Env-specific IgG in regular blood samples taken during the vaccination and pregnancy and vaccination is continued until high titres of IgG are detected. Immediately following calving, the first colostrum is collected by a registered veterinarian and calves are given their essential colostrum, leaving around one litre of colostrum per cow.

Following storage of 200 mls of whole colostrum, the remainder is fractionated and purified using methods to yield 1 kg of pure freeze dried antibodies. Whole colostrum and purified antibodies are assessed for breadth and titre of neutralizing antibody activity using a Env-pseudotyped reporter virus assay. Typical target cells are tested in these assays, as well as primary cells. HIV neutralisation is also confirmed in the PBMC spreading infection system.

Neutralisation assays for SIV to assess if this robust challenge model can be used for primate studies are also examined. Importantly, antibodies are titrated into pooled seminal plasma that is by-product from IVF clinical procedures, and into vaginal washings collected at various stages of the menstrual cycle and tested for neutralising activity. Usually the pH in the vagina is acid (between pH 4 and 5) but in the presence of semen the pH is increased to a neutral (pH 7) level. The activity of bovine colostrum antibodies are tested across this pH range.

### Example 4: Mechanisms of HIV Inhibition by Bovine Colostrum IgG in Viral Transcytosis and Neutralisation Assays In Vitro and in a Cervical Explant Infection Model

One major path by which HIV circumvents the host defence mechanisms is the transport of the virus within a protective vesicle across the interior of epithelial cells that face the inside of the vagina (known as transcytosis) without infecting these cells. Anti-transcytosis activity of bovine polyclonal antibodies are assessed in Hec 1-B cells using an EVOM2 Epithelia tissue voltohmmeter in a transcytosis assay. Whole colostrum, in addition to purified IgA and IgG are examined for anti-transcytosis activity. Cervical tissue is obtained, and the penetration of fluorescently labelled bovine IgG into the epithelial layers of the ectocervix, endocervix and columnar epithelium of the vagina is examined. HIV virion labelled with fluorescent Vpr is added to track the movement of HIV on and through these tissues in the explant culture model to observe virolysis or entrapment.

### Example 5: Formulation of Colostrum Neutralizing Antibodies into a Microbicide Gel and Testing Gel Safety and Efficacy in Rabbits

The most potent HIV-1 polyclonal bovine anti-Env antibodies (including anti-SOSIP gp140 antibodies) are tested and pooled and formulate into various water-based buffering gels.

Several formulations of anti-Env Ab (including anti-SOSIP gp140 antibodies) are prepared, including formulations that add back lactoferrin as a protein excipient, because it also has potent neutralising activity, and a casein and calcium carbonate formulation to test the possibility of retaining IgG binding activity after passage through the stomach and alimentary system for an oral delivered rectal microbicide.

The activity of antibodies when coformulated into different existing gels, such as glycol based K-Y lubricant gel, or the dendrimer microbicide gel, Vivagel, developed by Starpharma. Formulations that retain or enhance the breadth and potency of HIV neutralisation are tested for activity under neutral as well as acid pH conditions in vitro and their stability. The stability, biodistribution and reactogenicity/inflammation induced by the bovine antibodies are tested in rabbit toxicology studies. Biodistribution of bovine IgG are examined by histopathology, immunohistocehmistry, dermal observation of local inflammatory responses, immunogenicity by antibody and cellular responses to bovine IgG, and by Bioplex bead arrays or cytokine ELISA and EliSpot assay. Body weight, ophthalmology, ECG, clinical chemistry, haematology, urinalysis, organ weights and bone marrow and blood smears are tested for any abnormality. Control animals are given a placebo gel. Following cell toxicity studies, colostrum and purified Abs are co-cultivated with bacteria common in the vagina e.g. Lactobacillus acidophilus to assess the effect of colostrum Ab on the beneficial normal flora. Prior to primate challenge studies, the stability and activity of the formulation in the vaginal environment is tested at different time points following application to rabbits by recovering antibodies by saline washing.

### Example 6: Formulations

The following are formulated according to standard methods based on the following lists of ingredients. ‘Hyperimmune colostrum’ is hyperimmune colostrum containing antibodies to Env (including anti-SOSIP gp140 antibodies).

Formulation 1

A vaginal cream formulation is prepared by mixing the components listed in Table 2 below. For each application, 1-4 grams of the cream are vaginally administered with a suitable applicator such as a syringe.

TABLE 2

| Component | Weight Percent |
|---|---|
| Hyperimmune colostrum | 10-40 |
| Cetyl esters wax | 1-15 |
| Cetyl alcohol | 2-5 |
| White wax | 5-20 |
| Glyceryl monostearate | 10-30 |
| Propylene glycol | 10-15 |
| monostearate | |
| Methyl stearate | 5-90 |
| Benzyl alcohol | 3-10 |
| Sodium lauryl sulfate | 0.5-2.5 |
| Glycerin | 5-30 |
| Mineral oil | 0.1-95 |

Formulation 2

A vaginal cream formulation is prepared by mixing the components listed in Table 3 below. For each application, 1-4 grams of the cream are vaginally administered with a suitable applicator such as a syringe.

TABLE 3

| Component | Weight Percent |
|---|---|
| Hyperimmune colostrum | 10-40 |
| edetate disodium | 0.01-0.10 |
| glyceryl | 0.5-10 |
| monoisostearate | |
| methyl paraben | 0.18-0.20 |
| mineral oil | 0.1-95 |
| polyglyceryl-3-oleate | 2-3.5 |
| propylene glycol | 5-15 |
| propyl paraben | 0.02-0.10 |
| colloidal silicon dioxide | 1-5 |
| sorbitol solution | 2-18 |
| purified water | 10-20 |
| microcrystalline wax | 2-20 |

Formulation 3

A vaginal gel formulation is prepared by mixing the components listed in Table 4 below. For each application, 4 grams of the gel are vaginally administered with a suitable applicator such as a syringe.

TABLE 4

| Component | Weight Percent |
|---|---|
| Hyperimmune colostrum | 10-40 |
| Carbomer 934P | 0.1-0.5 |
| Edetate disodium | 0.01-0.10 |
| Methyl paraben | 0.18-0.20 |
| Propyl paraben | 0.02-0.10 |
| Propylene glycol | 5-15 |
| Sodium hydroxide | 0.01-0.05 |

Formulation 4

A rectal foam formulation is prepared by mixing the components listed in Table 5 below and inert propellants isobutene and propane. The foam is supplied in a aerosol container with a rectal applicator. For each application, 900 milligrams of the foam are rectally administered using the applicator.

TABLE 5

| Component | Weight Percent |
|---|---|
| Hyperimmune colostrum | 10-40 |
| Propylene glycol | 5-15 |
| Emulsifying wax | 10-15 |
| Polyoxyethylene- | 10-0.1-0.5 |
| stearyl ether | |
| Cetyl alcohol | 2-5 |
| Methyl paraben | 0.18-0.20 |
| Propyl paraben | 0.02-0.10 |
| T ethanolamine | 2-4 |
| Purified water | 10-30 |

### Example 7: Antigen Production, Purification and Vaccination of Cows

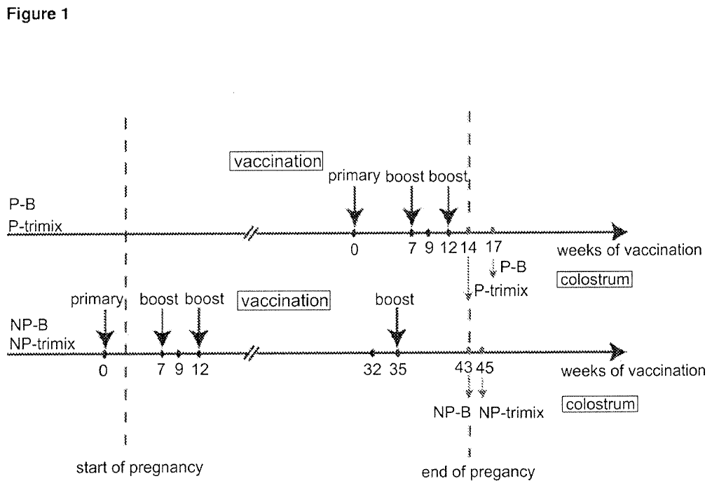

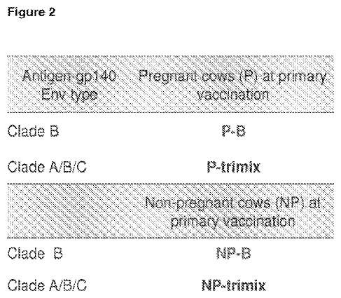

Soluble trimeric clade A (92UG8037.8; UG8), clade B (AD8) and clade C (93MW965.26; MW) HIV-1 Env gp140 were purified by lentil lectin affinity chromatography and gel filtration and 100 μg of gp140 in a proprietary adjuvant was used for three intramuscular vaccinations of 4 cows; 2 cows after conception (p) and two cows before conception (NP) according to the vaccination schedule shown in FIGS. 1 and 2.

### Example 8: IgG from Serum and Colostrum Binds to Gp140 Env of Clade A, B and C

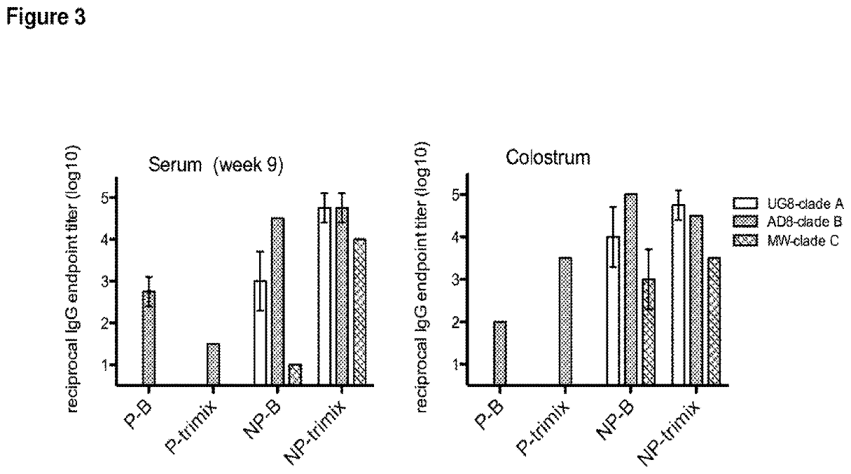

FIG. 3 shows the Env gp-140-specific IgG titres in serum 9 weeks following primary vaccination and colostrum determined by direct ELISA against gp140 Env of clade A (UG8), clade B (AD8) and clade C (MW). Reciprocal endpoint titres were determined using a 2 times OD cut-off based on pre-bleed samples or non-immune colostrum respectively. These results demonstrate IgG from serum and colostrum binds to gp140 Env of clade A, B and C. The results also demonstrate IgG from non-pregnant cows have broad binding activity to gp140 of clades A, B and C.

### Example 9: Purified Colostrum IgG from Non-Pregnant Cows Retains Binding to gp140 Env

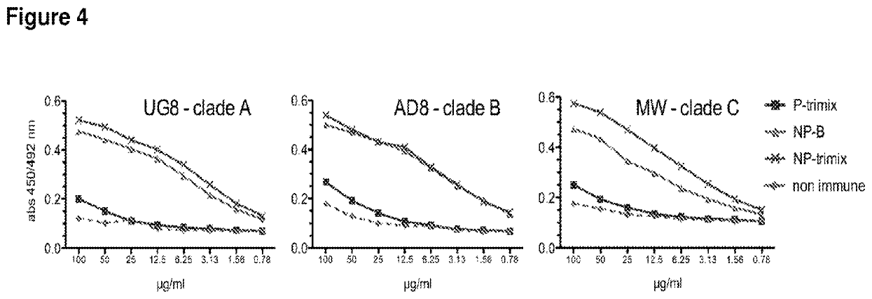

FIG. 4 shows the specific binding activity of purified colostrum IgG determined by direct ELISA against clade A (UG8), clade B (AD8) and clade C (MW) and absorbance (abs) measurement at 450 nm. These results demonstrate purified IgG from non-pregnant cows retains binding to gp140. The results also demonstrate purified IgG from non-pregnant cows retains broad binding activity to gp140 of clades A, B and C. The results also demonstrate cross-clade (heterologous) binding, with colostrum from nonpregnant cows vaccinated with clade B soluble Env gp140 oligomers binding gp140 of clades A B and C.

### Example 10: Bovine IgG Blocks Binding of Monoclonal Ab b12 to CD4 Binding Site of gp140

Several broadly neutralizing human monoclonal antibodies (MAbs) have been derived from infected individuals, including immunoglobulin GI (lgGl) b12 and 2G12. Among the most potent, the well-known b12 monoclonal antibody (Ab b12) occludes the site of CD4 binding on gp120 (which forms part of gp140) and prevents virus attachment to CD4 on target cells and is able to neutralize primary HIV-1 isolates. 2G12 recognizes a cluster of high mannose glycans on the viral envelope glycoprotein gp120. An understanding of the specificity of b12 binding, neutralization, and protection should aid in the development of immunogens that induce neutralizing antibodies of a similar specificity.

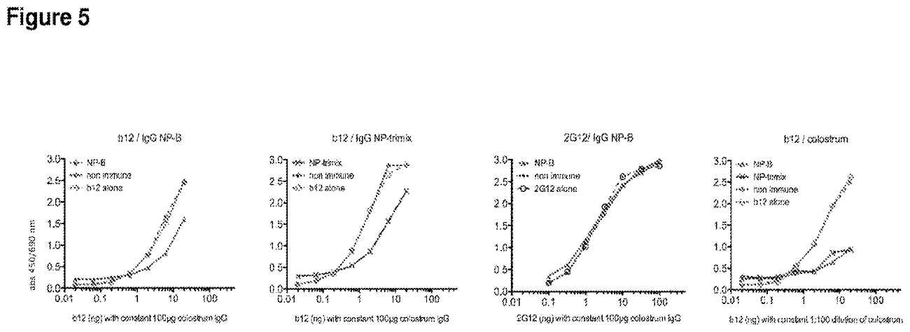

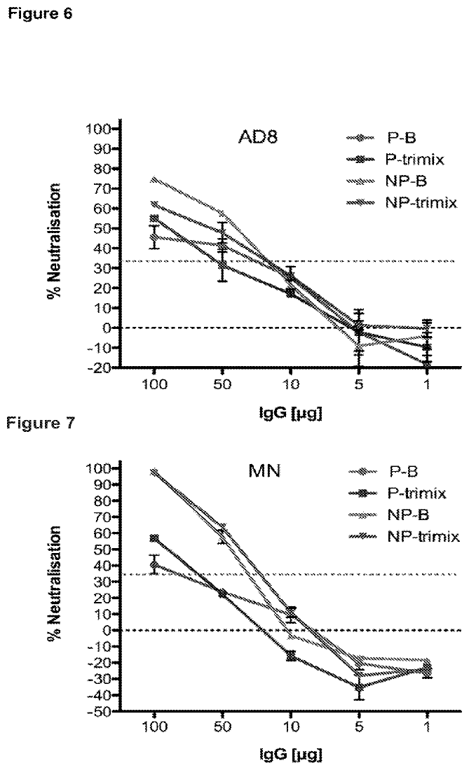

FIG. 5 shows bovine colostrum IgG competes with human neutralizing mAb b12 for binding at gp140 CD4 binding site. Competition ELISAs were performed by titrating b12 and 2G12 in a constant background of 100 pg IgG or a 1:100 dilution of whole colostrum. The ability of b12 and 2G12 to bind to AD8 (clade B gp140) in the presence or absence of colostrum IgG was detected by anti-human IgG HRP conjugated antibody. The results demonstrate bovine IgG blocks binding of the potent b12 antibody to the CD4 binding site of gp140 Env. The results also demonstrate bovine IgG from nonpregnant cows blocks binding of the potent b12 antibody to the CD4 binding site of gp140 Env.

### Example 11: Colostrum from Pregnant Cows Vaccinated with Clade A/B/C Gp140 and Non-Pregnant Cows Vaccinated with Clade B Gp140 have Broad Neutralizing Activity

Table 6 shows the neutralization profile of whole colostrum. Numbers represent percent neutralization for a 1:16 dilution against the indicated EGFP Env-pseudotyped viruses including common lab strains and the NIH clade B and C reference panel (ARRP #1 1227, #1 1326) in CF2 cells. Data shown is a representative experiment from two independent experiments. The results demonstrate colostrum from pregnant cows vaccinated with clade A/B/C gp140 and non-pregnant cows vaccinated with clade B gp140 have broad neutralizing activity. In particular, this neutralization is cross-clade (heterologous) neutralization, with colostrum from non-pregnant cows vaccinated with clade B soluble Env gp140 oligomers neutralizing HIV of clades A B and C. Furthermore, colostrum from pregnant cows vaccinated with trimix soluble Env gp140 oligomers neutralizing HIV of clades A B and C. The results also show that non-immune colostrum has neutralizing activity.

TABLE 6

| | | % Neutralization | | | | |
|---|---|---|---|---|---|---|
| PSV/Env clone | Clade | P-B | P-trimix | NP-B | NP-trimix | non-immune |
| AD8 | B | 59.1 | 78.9 | 74.3 | 10.6 | 64.0 |
| MN | B | 27.8 | 54.1 | 92.4 | 73.3 | 31.0 |
| SF162 | B | 43.3 | 75.2 | 68.9 | 33.6 | 30.9 |
| NI. 4.3 | B | 66.2 | 77.1 | 89.1 | 29.9 | 49.7 |
| 89.6 | B | 48.5 | 77.9 | 75.0 | 38.2 | 30.1 |
| 6535 clone 3 SVPB5 | B | 51.6 | 63.7 | 81.7 | 47.0 | 37.3 |
| QH0692 clone 42 (SVPB6) | B | 55.8 | 89.7 | 88.1 | 6.7 | 43.4 |
| PVO clone 4 SVPB11 | B | 25.5 | 66.6 | 92.9 | 25.0 | 25.2 |
| TRO clone 11, SVPB12 | B | 30.4 | 66.9 | 77.7 | 33.7 | 34.7 |
| Ac10.0 clone 29 SVPB13 | B | 25.5 | 63.1 | 79.5 | 15.6 | 26.3 |
| pWITO4160 clone33 | B | 37.2 | 53.6 | 83.8 | 40.7 | 31.2 |
| pTRJO4551 clone 16 | B | 4.8 | 52.3 | 89.4 | 31.1 | 15.5 |
| pREJO4541 clone 67 | B | 15.2 | 63.1 | 79.5 | 15.6 | 26.3 |
| pRHPA4259 clone 7 | B | 12.6 | 65.4 | 93.3 | 65.8 | 13.6 |
| SC422661.8 (SVPB8) | B | 5.7 | 61.7 | 93.3 | 26.1 | 26.5 |
| DU 156.12 | C | 16.2 | 43.7 | 42.8 | 26.7 | 28.6 |
| DU 172.17 | C | 41.3 | 73.8 | 68.9 | 19.0 | 54.0 |
| ZM 197M.PB7 | C | 29.7 | 52.3 | 73.1 | 41.8 | 41.8 |
| ZM 214M.PL15 | C | 8.2 | 50.0 | 67.7 | 32.4 | 36.2 |
| ZM 233M.PB6 | C | 33.5 | 61.7 | 77.4 | 49.2 | 30.3 |
| ZM 249M.PL1 | C | 30.1 | 51.0 | 83.5 | 19.6 | 36.5 |
| ZM53M.PB12 | C | 25.1 | 50.2 | 88.3 | 30.6 | 23.7 |
| ZM109F.PB4 | C | 19.7 | 47.7 | 81.5 | 28.8 | 22.2 |
| ZM135M.PL10a | C | 32.3 | 80.1 | 86.9 | 4.1 | 30.1 |
| CAP45.2.00.G3 | C | 34.5 | 33.7 | 57.9 | 28.5 | 37.2 |
| CAP210.2.00.E8 | C | 50.0 | 55.1 | 87.0 | 40.8 | 50.0 |
| 966 | A/E | 10.8 | 30.8 | 94.6 | 27.6 | 13.7 |

Neutralisation profile: green 0%-25%, yellow 25%-50%; orange 50%-75%; red 75%-100%.

### Example 12: Purified Colostrum IgG has Neutralizing Activity

FIG. 7 shows the neutralizing activity of colostrum purified IgG from all 4 vaccinated cows for 2 EGFP Env-pseudotyped reporter viruses (clade B). The neutralization characteristic of En-pseudotyped viruses; AD⁺; resistant, MN, sensitive.

Finally, it is understood that various other modifications and/or alterations may be made without departing from the spirit of the present invention as outlined herein.

Future patent applications may be filed on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or redefine the invention or inventions.

### Example 13: Production of Hyperimmune Colostrum Containing Polyclonal Anti-Env Antibodies Using SOSIP Gp140 as Antigen

Step 1—Production of vaccine for dairy cattle

The procedures for preparing antigen reported in Pub. No. WO/2004/078209 International Application No. PCT/AU2004/000277 (the contents of which are herein incorporated by reference) are used.

Step 2—The procedures for preparing antibodies from vaccinated cattle reported in Pub. No. WO/2004/078209 International Application No. PCT/AU2004/000277 (the contents of which are herein incorporated by reference) are used.

### Example 14: Production of Polyclonal Antibodies Binding to HIV Env, and Demonstration of Neutralization

Soluble SOSIP gp140 oligomers are prepared from clade A, B, and C HIV-1 strains from HeLa and/or 293T cells and are purified by lentil lectin affinity and gel filtration chromatography.

As an exemplary method BG505 SOSIP.664 gp140, BG505 SOSIP.664-His gp140, and BG505 SOSIP.664-avi gp140 may be expressed in HEK293F cells transfected using 293Fectin (Invitrogen) in the presence of Env plasmid and furin plasmid. The supernatants are purified on a lectin column and bound material eluted with MMP. After a buffer exchange with PBS, Avi-tagged trimers are biotinylated using BirA enzyme.

The purified Env proteins are further purified using Superose 6 10/300 GL size exclusion chromatography.

Four cows (two pregnant in second semester and two initially non-pregnant) are vaccinated with 100 g of purified HIV-1 SOSIP gp140 oligomer formulated with Montanide adjuvant. Two groups of two cows (one pregnant and one nonpregnant) are vaccinated with either clade B (AD8) only or with equal amounts (33.33 Mg) of clade A, B and C SOSIP gp140 (UG8, AD8 and MW) (referred to as ‘trimix’). All four cows receive at least three vaccinations whereas the last vaccination is given four weeks before giving birth. All four cows are proposed to seroconvert in under 10 weeks.

HIV-immune bovine colostrum is collected and pasteurised postpartum from all cows. Western blot analysis is used to confirm that colostrum IgG of all four cows is specific against HIV-1 Env gp140. Unfractionated colostrum is tested for neutralising activity in a HIV-1 Env-pseudotyped reporter virus assay. Clade A E, clade B and clade C pseudotype viruses including the NIH reference panel for clade B and C viruses are tested (total n=27) and compared with non-immune bovine colostrum that already has intrinsic infection-blocking activity due to lactoferrin and other bioactive peptides. Unfractionated colostrum from the trimix cow vaccinated during pregnancy is proposed to show high neutralisation of up to 50% for all B clade pseudoviruses as well as for the majority of C clade and clade A E pseudoviruses.

### Example 15: Polyclonal Neutralising Antibodies to HIV-1 SOSIP Gp140 from Bovine Colostrum

Twelve BSE-free pregnant cows are housed in an approved quarantine farm and vaccinated with 100 g of an eqimolar mix of four HIV-1 SOSIP gp140 oligomers: The SOSIP gp140 are formulated with adjuvant (Montanide) and administered twice before pregnancy and at least twice at 3-week intervals during the second trimester of pregnancy by a registered veterinarian.

An Env ELISA assay and Western blotting are used to monitor the levels of SOSIP gp140-specific IgG in regular blood samples taken during the vaccination and pregnancy and vaccination is continued until high titres of IgG are detected. Immediately following calving, the first colostrum is collected by a registered veterinarian and calves are given their essential colostrum, leaving around one litre of colostrum per cow.

Following storage of 200 mls of whole colostrum, the remainder is fractionated and purified using methods to yield 1 kg of pure freeze dried antibodies. Whole colostrum and purified antibodies are assessed for breadth and titre of neutralizing antibody activity using a Env-pseudotyped reporter virus assay. Typical target cells are tested in these assays, as well as primary cells. HIV neutralisation is also confirmed in the PBMC spreading infection system.

Neutralisation assays for SIV to assess if this robust challenge model can be used for primate studies are also examined. Importantly, antibodies are titrated into pooled seminal plasma that is by-product from IVF clinical procedures, and into vaginal washings collected at various stages of the menstrual cycle and tested for neutralising activity. Usually the pH in the vagina is acid (between pH 4 and 5) but in the presence of semen the pH is increased to a neutral (pH 7) level. The activity of bovine colostrum antibodies are tested across this pH range.

### Example 16: Extended Bovine Studies Using Purified Gp 140 Oligomers as Antigen

The studies of this Example were aimed to analyze the binding strength and breadth of the immune response induced by different Env vaccines in an effort to identify highly responding cows and find correlates between vaccinating Env and binding quality. A further aim was to investigate whether initial vaccination with the covalently-stabilized first-generation KNH1 SOSIP cleaved Env gp140 trimers and revaccination with covalently-stabilized second-generation BG505 SOSIP antigen could further enhance pre-existing immunity, especially in terms of the breadth of binding for Env gp140 from different HIV strains. Finally, this study also showed how CD4bs targeting was changed following revaccination with certain covalently-stabilized Env gp140, in particular 100 μg doses of BG505 SOSIP gp140 and AD8-6R SOSIP gp140.

Material and Methods

General Reagents

All materials used in these experiments were of analytical grade and were supplied by Sigma Aldrich, AbD Serotec, Thermo Scientific, Millipore, Emsure and Costar. Buffers were prepared in-house with purified Milli-Q water.

Blocking Buffer was made by dissolving 5% w/w of Casein Salt in PBS by heating the mixture to 70° with a heated magnetic stirrer for 3 hours or until the solution had clarified.

Dilution Buffer was made by 1/10 dilution of Blocking Buffer in PBS-Tween (0.1% Tween)

Coating Buffer was produced by diluting Tris and NaCl in Milli-Q water for a final concentration of 20 mM of Tris and 100 mM of NaCl at 8.8 pH

Tetramethybenzidine (TMB) substrate was made by dissolving 3,3′-5,5′ Sigma Aldrich T5525 tablets in 1 ml of DMSO with vigorous vortexing. 10 ml of Phosphocitrate buffer was added along with 2 ul of 30% Hydrogen Peroxide. TMB substrate was used immediately after preparation.

Stop Solution was made by dilution of 10M HCl with Milli-Q water, and pH adjusted to 1 with a pH meter.

Capture and Detection antibodies were stored at a ½ concentration in glycerol at 4° C. and diluted to final concentrations with Dilution Buffer immediately prior to use.

Production of G140 Env and Vaccination of Cows

Previously, uncleaved gp140 Env of several HIV clades was produced as described in Center RJ. Vaccine. 27(42):6605-12). All Env were water soluble forms truncated at the membrane proximal external region (MPER) and of trimeric structure. Vectors for clade B AD8, clade C MW, clade A KNHISOS-IP and clade B PSC89 were stably transfected into HeLa cells. Supernatant was harvested and gp140 trimers extracted through lentil-lectin affinity chromatography and size exclusion chromatography. Later, clade B AD8 SOS-IP was similarly produced in-house while clade A BG505 SOS-IP and BG505 SEKS gp140 was obtained from an external source. All gp140 proteins were stored in PBS+0.03% Sodium Azide to inhibit bacterial contamination.

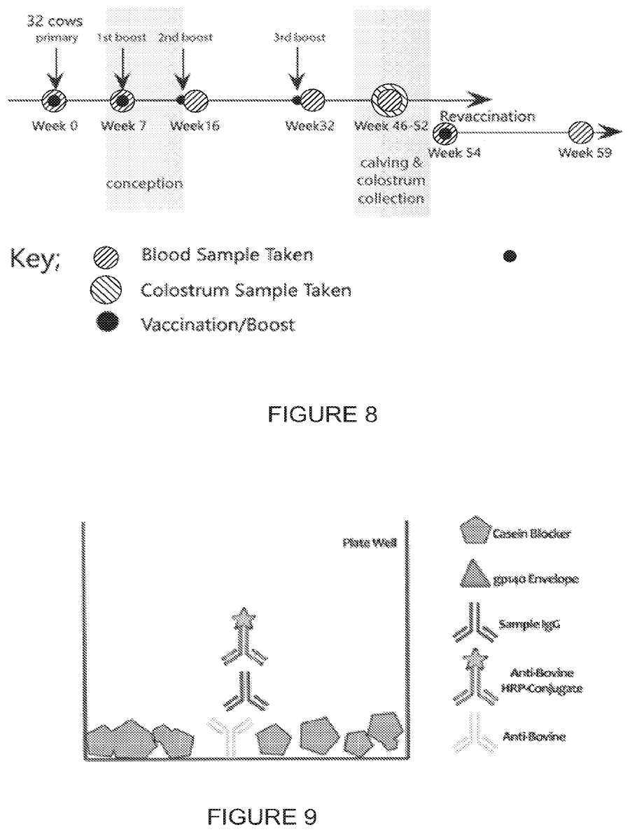

Reference is made to FIG. 8. By way of overview, the vaccination program was carried in two parts over the course of 60 weeks. The first part consisted of all cows being vaccinated simultaneously 4 times during the first 32 weeks. Cows were split into 5 vaccination groups which were differentiated by the clade or dose of vaccinating antigen. Black circles represent vaccination times. A single batch of colostrum was collected after calving (represented by the blue circle). In the second part of the study 10 high responding cows were then enrolled in a further revaccination study where they were boosted with a different Env antigen. Red circles represent dates when blood samples were taken. Blood was collected from 5 time points for all cows, and 7 for cows who also participated in the revaccination study. The light blue box represents the time period when cows became pregnant, while the red box represents the range of dates when cows gave birth.

Considering now the vaccination program in greater detail, the program consisted of two parts; an initial gp140 cow hyper-immunization study and a SOS-IP revaccination extension study.

In the initial cow hyperimmunisation study 32 female Holstein Friesian cattle (Bos Taurus) were randomly sorted into 5 groups. Each group was differentiated solely by the vaccination antigen. Animals in the same group received the same strain and dose of Env for the entire course of the initial study. Each vaccination consisted of a 2 ml injection containing 1 ml of purified gp140 oligomers in PBS solvent and 1 ml of Seppic Montanide (ISA206) adjuvant.

The 5 vaccination groups were differentiated by the antigens with which they were vaccinated with. 8 cows (2150, 9533, 35, 8434, 647, 8203, 23, 537) being sorted into a group which received 500 μg of clade B AD8 Env for each vaccination. 6 cows (623, 6714, 2005, 3096, 9516, 9511) received 100 μg of clade B AD8 Env. 2 cows (609, 617) were vaccinated with 100 μg of clade B KNHISOS-IP. 8 cows (5682, 7333, 3333, 9506, 9552, 2223, 26, 9545) received 500 μg of clade B PSC89. Finally, the last 8 cows (5641, 9540, 2036, 657, 698, 5586, 9244, 2179) received 500 μg of clade C MW Env. During this part of the study 5 cows failed to complete the study; cows 9511 and 7333 delivered prematurely. Cow 26 aborted and produced no colostrum. Cows 9545 and 698 died from other complications.

Cows were vaccinated at weeks 0, 7, 16 and 32. Blood and Serum samples were collected from cows at weeks 0, 7, 17, 33. During weeks 46-52 serum, blood and colostrum samples were collected on a per cow basis on the day they gave birth. The vaccination schedule is summarized in FIG. 8.

The second part of the vaccination study began approximately 1 week after the last cow had given birth. 10 cows took part in this further 5-week long extension study where they were vaccinated with an additional round of Env. Cows were vaccinated with 50 μg or 100 μg of BG505 SEKS, BG505 SOS-IP, AD8 SOS-IP or AD8. Table 7 below summaries the vaccination strategy of all cows for both parts of the study.

| Cow ID# | Vaccination Env | Revaccination Env |
|---|---|---|
| 9533 | AD8 500 | 50 μg BG505 SEKS gp140 |
| 537 | AD8 500 | 50 μg BG505 SEKS gp140 |
| 35 | AD8 500 | 50 μg BG505 SOS-IP gp140 |
| 8434 | AD8 500 | 100 μg AD8 6R SOS-IP 664 6H gp140 |
| 2150 | AD8 500 | |
| 647 | AD8 500 | |
| 8203 | AD8 500 | |
| 1134 | AD8 500 | |
| 2005 | AD8 100 | 100 μg AD8 gp140 |
| 623 | AD8 100 | |
| 6714 | AD8 100 | |
| 3096 | AD8 100 | |
| 9516 | AD8 100 | |
| 9511 | AD8 100 | |
| 609 | KNH1 SOS-IP | 100 μg BG505 SOS-IP gp140 |
| 617 | KNH1 SOS-IP | 50 μg BG505 SOS-IP gp140 |
| 5586 | MW 500 | 100 μg AD8 6R SOS-IP 664 6H gp140 |
| 9244 | MW 500 | 50 μg BG505 SOS-IP gp140 |
| 5641 | MW 500 | |
| 9540 | MW 500 | |
| 2036 | MW 500 | |
| 657 | MW 500 | |
| 698 | MW 500 | |
| 2179 | MW 500 | |
| 9506 | PSC89 500 | 50 μg BG505 SOS-IP gp140 |
| 5682 | PSC89 500 | |
| 7333 | PSC89 500 | |
| 3333 | PSC89 500 | |
| 9552 | PSC89 500 | |
| 2223 | PSC89 500 | |
| 26 | PSC89 500 | |
| 9545 | PSC89 500 | |

As will be noted from Table 7 above, the vaccination program consisted of two parts. All 32 cows took part in the initial vaccination study and their vaccinating antigen is detailed in the 2nd column. The extension revaccination study is detailed in the 3rd column. Not all 32 cows participated in the extension study. The 10 cows which did participate in the extension study are indicated above by the shaded table cells.

Storage and Preparation of Samples

Serum was taken at week 0 and week 7 on the same day as the vaccinations. The 3rd serum samples were taken 1 week after the 3rd injection, while the 4th serum samples were taken 5 to 6 days after the 4th injection. On the day of calving, colostrum samples were collected within six hours of birth by milking and immediately frozen at −20° C. Serum and Full Blood samples were also taken at this time and similarly frozen.

Colostrum samples were then defatted, pasteurized and stored. In summary a portion of colostrum was unfrozen and centrifuged at 10000 g for 30 minutes at 4° C., pasteurized at 63° C. and then centrifuged again at 10000 g for 10 minutes. Colostrum pH was then lowered to 4.6 via mixing with sodium acetate at room temperature. Samples were centrifuged again and frozen at 20° C. until needed.

A portion of processed colostrum was taken aside and further processed for IgG purification. Colostrum whey was dialyzed against PBS using a 30-kDa ultrafiltration membrane (Amicon Ultra 15 ml; Millipore). IgG was purified using a protein G column (GE Healthcare). Purified IgG was filter sterilized and concentration measured with a spectrophotometer at 280 nm (Thermo Scientific ND2000).

On the day of ELISA analysis, colostrum samples were thawed, resuspended and then centrifuged at 20,000 g for 5 mins, using a benchtop microcentrifuge. This was done to remove trace precipitate. After centrifugation, a sample of each colostrum supernatant was diluted by 1/10 with Dilution Buffer and then mixed and centrifuged again at 20,000 g for 5 mins to remove any remaining precipitate. The supernatant from the 1/10 diluted colostrum was then further diluted to prepare the first 1/100 dilution of the colostrum for ELISA assays.

Blood samples were frozen at −20° C. immediately after collection. Upon first use, samples were aliquoted into smaller 50 ul tubes and a mixed with thimerosal for a final concentration of 0.01% to inhibit bacterial growth. Opened blood samples were stored at 4° C. to prevent freeze-thaw induced antibody degradation.

Quantitative Elisa

Quantitative ELISA was performed to measure and compare the concentration of polyclonal IgG in the serum and colostrum samples. Apart from natural animal to animal variation, serum antibody levels have been reported to be lowered during late pregnancy.

Reference is made to FIG. 9. In brief, 96-well Costar polyvinyl plates were coated with 100 ng/well (100 ul of 1 ug/ml) of mouse anti-bovine (AbD Serotec) monoclonal capture antibody in Coating Buffer and incubated at 4° C. overnight. The next day, plates were washed with PBST 4 times and PBS 2 times and then blocked with Blocking Buffer for 1 hour at 37° C. During this time, the detection antibody was prepared; rabbit anti-bovine monoclonal IgG (Sigma AG2G5) was diluted to a final concentration of 1/8000 in dilution buffer, and 1% v/v of normal mouse serum was added to minimize capture to detection antibody cross reactivity. The detection antibody mixture was then incubated at 37° C. for 3 hours. After wells were blocked and washed again, 100 ul of Serum or Colostrum samples were added to wells and 2-fold dilutions were made. Bovine IgG purified from colostrum whose concentration had been quantified by a Spectrophotometer (Thermo Scientific ND2000) used to construct the standard. Samples were incubated at 37° C. for 2.5 hours. During all incubation steps in these assays, plates were sealed to minimize evaporation and edge effects.

After another round of washing, 100 ul of the detection antibody mix was added to each well and incubated at room temperature for 1 hour. After a final washing, color was developed using 3,3′-5,5′-tetramethybenzidine (TMB) substrate for 15 minutes. The reaction was stopped with the addition of 100 ul of IM HCl. Absorbance was read on a Labsystems “Multiskan Ascent” ELISA plate reader at 450 nm against a reference of 690 nm.

Binding Assay

Binding assays were performed for several purposes;
To screen colostrum samples to identify highly-responding cows
To compare the strength and breadth of anti-Env antibodies raised by the different Env antigens
To study the effectiveness of the SOS-IP revaccination study in further boosting Env titer.

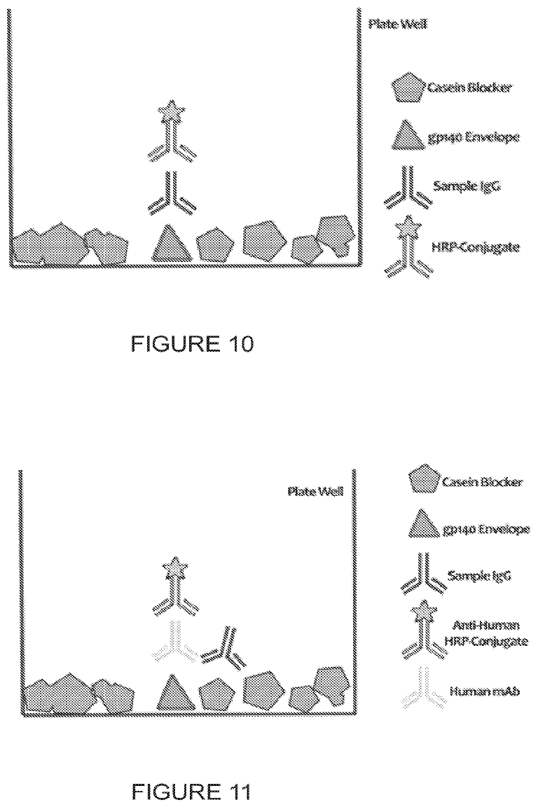

Reference is made to FIG. 10. In brief, 96-well Costar polyvinyl plates were coated with 100 ng/well (1 ug/ml) of purified soluble gp140 Envelope in Coating Buffer, plates were sealed and incubated overnight at 4° C. gp140 Envelope Strains used to coat the plates were;
AD8 (B-clade)
PSC89 (B-clade)
MW (C-clade)
BG505 SOS-IP (A-clade)

The next day, excess Env was removed by washing with PBST 4 times and PBS 2 times. Uncoated plastic in the plate wells was then blocked with 5% Casein Blocking Buffer for 1 hour at room temperature.

After incubation, plates were then washed again, and 146 ul of 1/100 concentration of Serum or Colostrum samples diluted in dilution buffer was added to the first well. Half log dilutions were made and the plates were incubated for 2.5 hours at room temperature. After a further washing, 100 ul of 1/2000 of horse-peroxidase conjugated rabbit anti-bovine IgG (diluted in dilution buffer) was added to each well, and incubated at room temperature for 1 hour. A final washing was made. Color reaction was developed using 3,3′-5,5′-tetramethybenzidine (TMB) substrate for 15 minutes. The reaction was stopped with 100 ul of IM HCl. Absorbance was read on a Multiskan Ascent at 450 nm against a reference of 690 nm to remove background.

Competition Assay

In order to study the epitope binding of polyclonal IgG, a competition ELISA against CD4 binding site (CD4bs) targeting monoclonal antibodies was performed. Previous work had suggested cows showed binding against the CD4bs neutralising epitope (50, 61). Prior to the competition assay, optical densities of the binding assays against AD8 gp140 Env were used to determine suitable concentrations for the samples which would lie within the linear range of detection.

Reference is made to FIG. 11. 96-well Costar polyvinyl plates were coated with 100 ng/well (1 ug/ml) of purified soluble AD8 gp140 Env in Coating Buffer and incubated at 4° C. overnight. After incubation, excess Env was washed off with PBST 4 times and PBS 2 times. Uncoated surface in the plate wells were then blocked by incubation with Blocking Buffer for 1 hour at room temperature. After another round of washing, 100 ul of Serum or Colostrum samples diluted in dilution buffer was added to wells and incubated at room temperature for 2 hours. After washing, 146 ng/well of VRC01 or b12 mAb was added to wells and half-log dilutions made. The human monoclonal competition antibodies were then incubated for 2 hours at room temperature. After washing, 100 ul of 1/1000 of HRP conjugated goat anti-human IgG (AbD Serotec) was added to each well and incubated at room temperature for 1 hour. After a final washing, color was developed using 3,3′-5,5′-tetramethybenzidine (TMB) substrate for 15 minutes. The reaction was stopped with the addition of 100 ul of IM HCl. Absorbance was read on a Labsystems “Multiskan Ascent” ELISA plate reader at 450 nm against a reference of 690 nm.

### Experimental Results

Colostrum Binding Assay Screen

This initial ELISA assay was performed to both identify highly responding cows and compare the binding activity of antibodies raised by different Env vaccines. Colostrum samples from all cows who calved successfully were tested against 3 different clades of Env (AD8, PSC89 and MW gp140). A minor alteration was made to the ELISA protocol with samples being diluted to a single concentration of 1/100. A positive signal was determined to be an optical density (OD) over double that of pre-immune serum IgG.

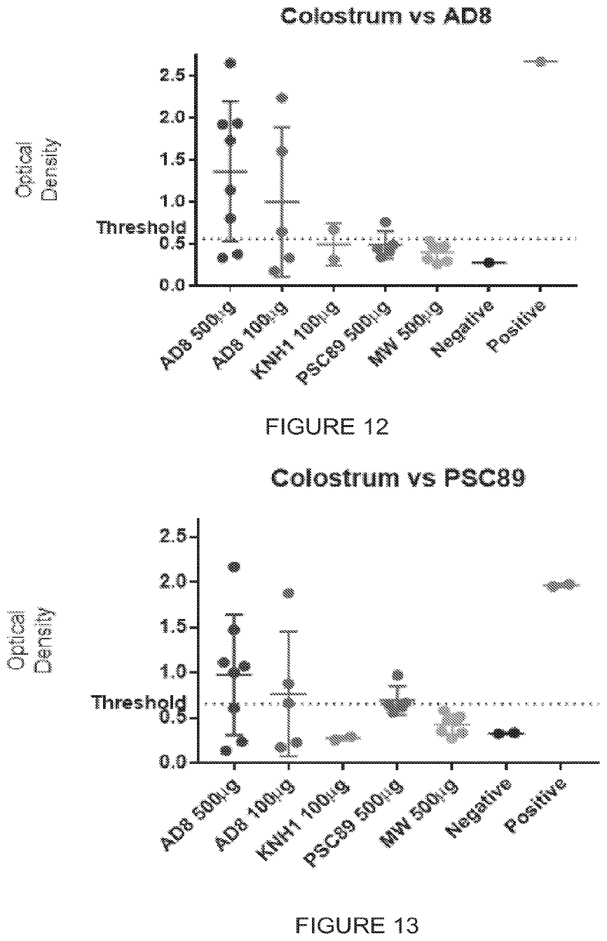

Results (FIG. 12) for AD8 gp140 Env binding show that 6 out of 8 of cows vaccinated with 500 μg of AD8 had an endpoint titer over 100. Similarly, 3 out of 6 cows vaccinated with 100 μg of AD8, 1 of 2 KNHISOS-IP 100 μg cows, 1 of 8 PSC89 500 μg cows and no MW 500 μg vaccinated cows had a colostrum endpoint titer over 100. The mean OD of the AD8500 μg vaccination group was 1.365±0.2937. The mean OD of the AD8 100 μg vaccination group was 1.00±0.39. The mean OD of the KNH1 100 μg vaccination group was 0.49±0.17. The mean OD of the PSC89 500 μg vaccination group was 0.49±0.071. Lastly, the mean OD of the MW 500 μg vaccination group was 0.41±0.040.

Having further regard to FIG. 12 points above the “Threshold” line represents samples with an endpoint titer above 100. Pooled pre-immune serum from the 32 cows present in the study was used as the negative. The positive control is the highest binding colostrum sample from a previous study. All points represent the mean of 2 replicates. Error bars for individual samples were smaller than the graphical representation of the points and not shown. The mean and standard deviation of each vaccination group are shown. Note only 27 samples were tested as 5 cows did not survive and/or calve to produce colostrum.

Results would be biased higher against the autologous vaccinating antigen, so samples were tested against non-autologous envelope as well. Against uncleaved PSC89 gp140 Envelop, results show (FIG. 13) 5 out of 8 of cows vaccinated with 500 μg of AD8 had an endpoint titer over 100. 3 out of 6 cows vaccinated with 100 μg of AD8, 0 of 2 KNHISOS-IP 100 μg cows, 2 of 5 PSC89 500 μg cows and 0/7 MW vaccinated cows had a colostrum endpoint titer over 100 against PSC89 gp140. The mean OD of the AD8 500 μg vaccination group was 0.97±0.23. The mean OD of the AD8 100 μg vaccination group was 0.76±0.30. The mean OD of the KNH1 100 μg vaccination group was 0.27±0.017. The mean OD of the PSC89 500 μg vaccination group was 0.69±0.071. Finally, the mean OD of the MW 500 μg vaccination group was 0.42±0.041.

Having further regard to FIG. 13, points above the “Threshold” line represents samples with an endpoint titer above 100. Pooled pre-immune serum from the 32 cows present in the study was used as the negative. The positive control is the highest binding colostrum sample from a previous study. All points represent the mean of 2 replicates. Error bars for individual samples were smaller than the graphical representation of the points and not shown. The mean and standard deviation of each vaccination group are shown. Note only 27 samples were tested as 5 cows did not survive and/or calve to produce colostrum.

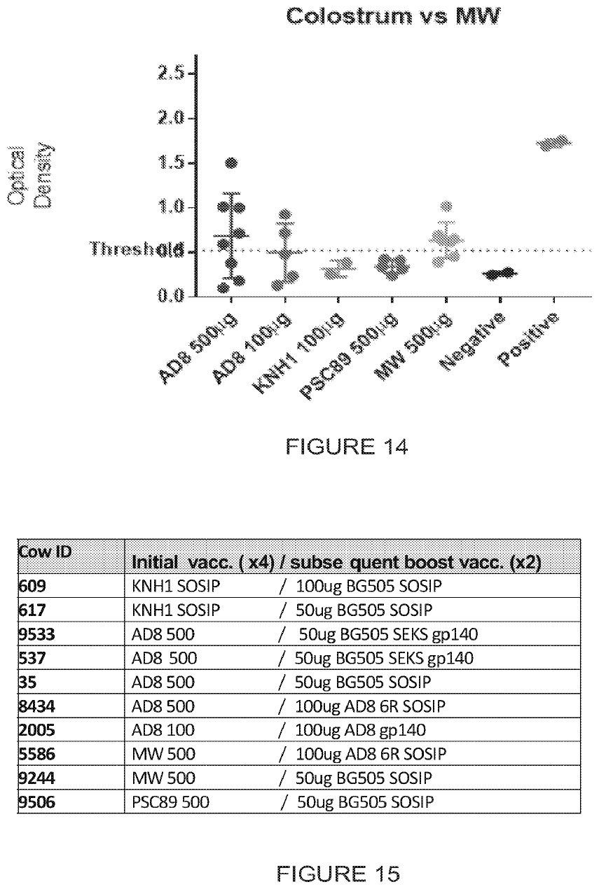

Against uncleaved MW gp140 Envelope, results (FIG. 14) show that 5 out of 8 of cows vaccinated with 500 μg of AD8 had an endpoint titer over 100. 2 out of 6 cows vaccinated with 100 μg of AD8, 0 of 2 KNHISOS-IP 100 μg cows, 0 of 5 PSC89 500 μg cows and 5/7 MW vaccinated cows had a colostrum endpoint titer over 100. The mean OD of the AD8 500 μg vaccination group was 0.68±0.16. The mean OD of the AD8 100 μg vaccination group was 0.49±0.14. The mean OD of the KNH1 100 μg vaccination group was 0.31±0.064. The mean OD of the PSC89 500 μg vaccination group was 0.34±0.032. The mean OD of the MW 500 μg vaccination group was 0.63±0.075.

Having further regard to FIG. 14, points above the “Threshold” line represents samples with an endpoint titer above 100. Pooled pre-immune serum from the 32 cows present in the study was used as the negative. The positive control is the highest binding colostrum sample from a previous study. All points represent the mean of 2 replicates. Error bars for individual samples were smaller than the graphical representation of the points and not shown. The mean and standard deviation of each vaccination group are shown. Note only 27 samples were tested as 5 cows did not survive and/or calve to produce colostrum.

These studies show that cows were more likely to show strong binding towards the autologous antigen they were vaccinated with. AD8 cows were most likely to show cross-breadth binding, and in many cases even exceeded the binding activity of cows vaccinated with the autologous antigen. AD8 vaccinated cows also had the colostrum with the greatest binding activity (in averaged OD).

Notably, the relative strength of binding remained consistent for samples within each vaccination groups throughout the assays, despite the change in Env being bound. Highly binding samples in the AD8 500 μg group were serum from cows; 2150, 35, 8434, 537, 647 and 9533. For the AD8 100 μg group, 2005, 537 and 1134 showed strong binding. 609 consistently showed stronger binding than 617 in the KNHISOS-IP group. 5586 and 9244 showed the strongest binding in the MW group. In the PSC89 group, only 2223 showed any, if even weak binding.

Following this screen, these highly responding cows' samples were focused on for further study.

| AD8 500 | AD8 100 | KNH1SOS-IP | PSC89 | MW |
|---|---|---|---|---|
| 2150 | 2005 | 609 | 2223 | 5586 |
| 35 | 9516 | | | 9244 |
| 8434 | 3096 | | | 5641 |
| 537 | | | | 2179 |
| 647 | | | | 657 |
| 9533 | | | | |

Having regard to Table 8 above, samples that showed an OD above the threshold in at least 1 assay are shown. Samples closer to the top of the table have a better rank within the same group. Ranking is based on averaged results from OD values from all assays against the 3 Env proteins.

Measurement of Antibody Concentration

During the last trimester of pregnancy, serum antibody concentrations decrease as serum IgG is concentrated into the colostrum. Highly responding cows were inducted into the revaccination study with a varying amount of time elapsing since giving birth, with late calving cows having up to 3 less weeks to recover. Quantitative ELISAs were performed to study if serum IgG levels had comparable concentrations and to study changes in serum IgG concentration over time.

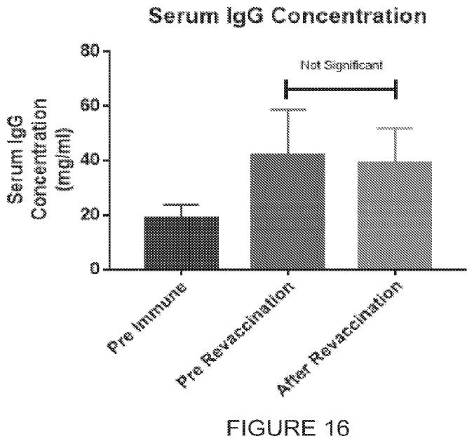

One-way ANOVA analysis of the IgG concentrations showed none of the results had a significant (P<0.05) increase in serum IgG after revaccination, suggesting all cows' IgG had recovered from pregnancy (see FIG. 16). Concentrations of IgG also showed similar variation to previous measurements in dairy cattle. Distribution of serum IgG concentrations was within range of natural animal to animal variation. The mean IgG (mg/ml) level of cows before any vaccination (pre-immune) was 19.1±4.74. The mean IgG level before revaccination was 42.1±16.5, and after revaccination was 38.8±14.05. Serum IgG had increased roughly 2 fold compared to before the cows were vaccinated. It was ultimately decided that serum samples would not be diluted or normalized prior to further assays.

| | | | | Sig? |
|---|---|---|---|---|
| Cow # | Pre-Immune | Pre-Revaccination | After Revaccination | > |
| 609 | 19.77 ± 1.58 | 43.18 ± 1.32 | 45.53 ± 5.68 | No |
| 9533 | 27.66 ± 0.75 | 59.56 ± 7.47 | 56.87 ± 12.4 | No |
| 8434 | 18.44 ± 3.71 | 50.44 ± 2.39 | 30.13 ± 7.89 | No |
| 9244 | 19.37 ± 1.09 | 23.83 ± 1.45 | 26.14 ± 2.45 | No |
| 617 | 18.61 ± 0.72 | 42.76 ± 2.84 | 25.79 ± 1.93 | No |
| 2005 | 25.87 ± 1.7 | 51.68 ± 5.73 | 50.08 ± 13.05 | No |
| 35 | 12.61 ± 0.18 | 20.42 ± 2.3 | 27.54 ± 1.44 | No |
| 537 | 13.69 ± 1.03 | 22.23 ± 0.7 | 26.06 ± 3.63 | No |
| 9506 | 16.1 ± 1.32 | 41.59 ± 4.91 | 36.78 ± 8.22 | No |
| 5586 | 18.92 ± 1.35 | 51.12 ± 11.36 | 62.89 ± 24.56 | No |

Table 9 above shows serum IgG concentration as measured by a quantitative ELISA. All units are in mg/ml. The results represent the mean of 3 replicates and error is represented by standard deviation. Samples were tested from the 10 cows who took part in the revaccination study. Each column represents a timepoint; on the day of the first vaccination (when cows were still pre-immune), on the day of revaccination with the SOS-IP antigen (approximately 1 week to a month after calving). The final column was taken 5 weeks after the revaccination.

Serum Binding Titres Before and after Revaccination

Following identification of highly responding animals with strongly binding colostrum and the confirmation of comparable serum IgG concentrations, the binding titers of serum samples were analyzed before and after revaccination. Previous indications were that extended vaccination (after a single pregnancy) with uncleaved AD8 failed to further increase the strength or breadth of the antibody response, thus studies were attempted to show whether or not revaccination with a different SOS-IP Env could do so in highly responding cows. Serum samples were tested before and after revaccination serum samples from cows against AD8, MW and BG505 SOS-IP. A positive result was determined as an optical density of over double the optical density of the negative sample (pooled pre-immune sera) diluted to the same concentration. Endpoint titers are shown below in Table 10.

| | AD8 | | MW 500 | | BG505 SOS-IP | |
|---|---|---|---|---|---|---|
| Cow | Pre- | After- | Pre- | After- | Pre- | After- |
| ID# | Revac | Revac | Revac | Revac | Revac | Revac |
| 9533 | 3160 | 31600 | 1000 | 3160 | 100 | 316 |
| 35 | 3160 | 10000 | <100 | 3160 | <100 | 100 |
| 8434 | 1000 | 31600 | 100 | 3160 | <100 | 1000 |
| 537 | 10000 | 3160 | 3160 | 1000 | <100 | <100 |
| 2005 | 1000 | 10000 | <100 | 3160 | <100 | 100 |
| 609 | <100 | 3160 | <100 | 316 | <100 | 3160 |
| 617 | <100 | 316 | <100 | <100 | <100 | 100 |
| 5586 | <100 | <100 | 3160 | 1000 | 100 | <100 |
| 9244 | 100 | 316 | 208 | 1000 | 100 | 316 |

The above Table shows reciprocal serum endpoint as measured by ELISA. Sample were tested against 3 clades of Env; AD8, MW and BG505 SOS-IP. Results represent the mean of 2 replicates. Dilutions below 1/100 were not tested. The first column per group represents the titer before revaccination, while the 2nd column represents the post-revaccination titer.

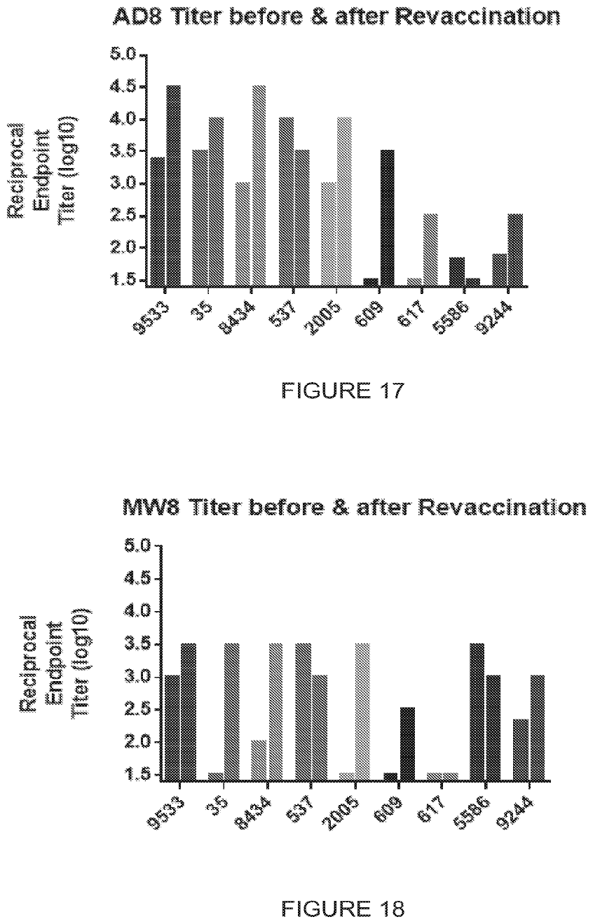

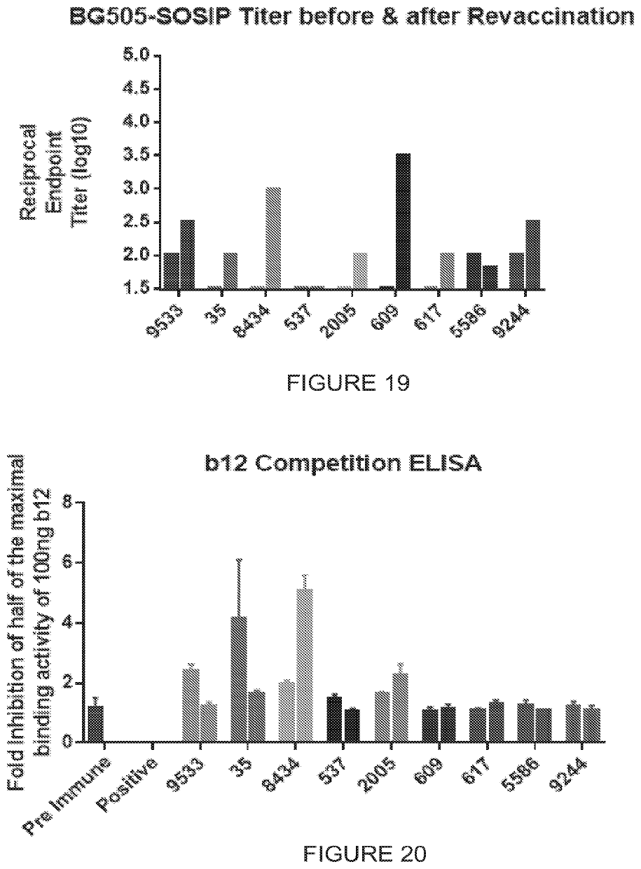

Reference is now made to FIGS. 17, 18, and 19. These Figures show reciprocal serum endpoint as measured by ELISA. Results show the endpoint titers from the last 2 columns of FIG. 17. Results represent the mean of 2 replicates and error bars (if any) represent standard deviation. The first column per group represents the titer before vaccination, and the 2nd column the post-revaccination titer.

Endpoint titers against AD8 and MW were in accordance to the colostrum ODs found earlier. AD8 cows showed the highest titers and cross-clade binding. Prior to revaccination only AD8 vaccinated cows reached titers over 3160 (Cows 9533, 35 and 8434 against autologous AD8), and only the AD8 vaccinated cow 537 had a MW titer as high (at 3160) compared to cows vaccinated with MW. This was not true for BG505 SOS-IP titers however, as only a single AD8 cow (9533) had a titer greater than 100, compared to two MW vaccinated cows.

| Cow | | MW | BG505 | | Revaccination |
|---|---|---|---|---|---|
| ID# | AD8 | 500 | SOS-IP | Start Env | Env |
| 9533 | 1 | 0.5 | 0.5 | AD8500 | 50 μg BG505 |
| | | | | | SEKS gp140 |
| 35 | 0.5 | >1.5 | >0 | AD8 500 | 50 μg BG505 |
| | | | | | SOS-IP gp140 |
| 8434 | 1.5 | 1.5 | >1 | AD8500 | 100 μg AD8 |
| | | | | | SOS-IP gp140 |
| 537 | −0.5 | −0.5 | ? | AD8 500 | 50 μg BG505 |
| | | | | | SEKS gp140 |
| 2005 | 1 | >1.5 | >0 | AD8 100 | 100 μg |
| | | | | | AD8 gp140 |
| 609 | >1.5 | >0.5 | >1.5 | KNH1 | 100 μg BG505 |
| | | | | SOS-IP | SOS-IP gp140 |
| 617 | >0.5 | ? | >0 | KNH1 | 50 μg BG505 |
| | | | | SOS-IP | SOS-IP gp140 |
| 5586 | ? | −0.5 | −0.5 | MW 500 | 100 μg AD8 |
| | | | | | SOS-IP gp140 |
| 9244 | 0.5 | 0.5 | 0.5 | MW 500 | 50 μg BG505 |
| | | | | | SOS-IP gp140 |

Table 11 above shows Log 10 fold change of titer and Vaccination Strategy of Tested Cows The increase of Env titers before and after revaccination was calculated by dividing the after revaccination titre with the before revaccination titer. Titers smaller than 100 were treated as a 100 and a “>” sign appended to the resultant figure.

Overall, SOS-IP revaccination enhanced the binding towards the initial autologous antigen, as well as increasing the breadth of binding towards non-vaccinating antigens. 5 out of 6 SOS-IP revaccinated cows showed an increase in binding titer. There was a strong correlation between changes in Env titer between different antigens, with decreases or increases in titer towards one strain of Env reflected in titer towards the other Env tested (FIG. 17). The greatest increases in titer were from SOS-IP revaccinated cows (8434, 609).

Competition ELISA Against CD4bs Antibodies

Previous studies herein established that a major neutralising epitope targeted by the cows' polyclonal antibodies was the CD4 binding site (CD4bs) of HIV Envelope. A competition ELISA was performed to determine whether anti-CD4bs antibodies had emerged in these cows, and if there was any correlation between CD4bs competition with binding titers and/or the SOS-IP revaccination. Thus, 9 of the revaccinated cows were competed against a human BrNAb known to bind the CD4 binding site (b12).

| Sample | Before | Significant? | After | Significant? |
|---|---|---|---|---|
| b12 only | 1 | | | |
| Pre-Immune | 1.18 ± 0.31 | | | |
| 9533 | 2.37 ± 0.26 | Yes | 1.22 ± 0.12 | No |
| 35 | 4.17 ± 1.93 | Yes | 1.62 ± 0.13 | Yes |
| 8434 | 1.98 ± 0.1 | Yes | 5.06 ± 0.54 | Yes |
| 537 | 1.48 ± 0.13 | Yes | 1.06 ± 0.08 | No |
| 2005 | 1.62 ± 0.07 | Yes | 2.27 ± 0.36 | Yes |
| 609 | 1.06 ± 0.13 | No | 1.15 ± 0.14 | No |
| 617 | 1.07 ± 0.08 | No | 1.29 ± 0.14 | No |
| 5586 | 1.26 ± 0.17 | No | 1.09 ± 0.03 | No |
| 9244 | 1.22 ± 0.17 | No | 1.11 ± 0.14 | No |

Table 12 above shows b12 was titrated against a constant background of 1/100 dilution serum. b12 binding was detected using goat anti-human HRP conjugated antibody. Inhibition was measured by calculating the fold change in b12 required to give an optical density equal to half of the maximum optical density of the b12 binding without any competition from colostrum. A higher result represents greater competition from the colostrum. Results represent the mean of two repeats, and error bars represent standard deviation. One-way ANOVA was used to compare samples against the pre-immune, and samples with a P<0.05 were treated as significant.

Inhibition activity (represented by fold change of b12 needed to halve the maximal binding) is summarized in FIG. 20. Of the 16 samples tested, 8 out of 8 samples which showed significant binding against CD4bs were from cows which had initially been vaccinated with an AD8 Env. The highest competing sample was cow #8434's serum after revaccination, a cow who was initially vaccinated with AD8 500 μg and was later revaccinated with AD8SOS-IP 100 μg. This suggests that AD8 alone can stimulate CD4bs antibodies.

Furthermore, these studies examined if the changes in CD4bs competition before and after vaccination were significant. CD4bs competition change appeared to be independent of the SOS-IP revaccination, as there was no solid correlation between SOS-IP revaccination and a significant positive change in fold inhibition. Cow 8434 (AD8 SOS-IP revaccination) saw a significant increase in CD4bs competition.

| | ΔFold | | P | Initial | Revaccination |
|---|---|---|---|---|---|
| Sample | Change | Sig? | Value | Vaccine | Vaccine |
| 9533 | −1.15 | Yes | <0.0001 | AD8 500 | 50 μg BG505 |
| | | | | | SEKS gp140 |
| 35 | −2.55 | Yes | <0.0001 | AD8 500 | 50 μg BG505 |
| | | | | | SOS-IP gp140 |
| 8434 | 3.08 | Yes | <0.0001 | AD8 500 | 100 μg AD8 |
| | | | | | SOS-IP gp140 |
| 537 | −0.42 | Yes | <0.0001 | AD8 500 | 50 μg BG505 |
| | | | | | SEKS gp140 |
| 2005 | 0.65 | Yes | <0.0001 | AD8 500 | 100 μg |
| | | | | | AD8 gp140 |
| 609 | 0.09 | No | 0.9532 | AD8 100 | 100 μg BG505 |
| | | | | | SOS-IP gp140 |
| 617 | 0.22 | No | 0.1573 | KNH1 | 50 μg BG505 |
| | | | | SOS-IP | SOS-IP gp140 |
| 5586 | −0.17 | No | 0.3589 | KNH1 | 100 μg AD8 |
| | | | | SOS-IP | SOS-IP gp140 |
| 9244 | −0.11 | No | 0.8726 | MW 500 | 50 μg BG505 |
| | | | | | SOS-IP gp140 |

Table 13 above shows change in Fold Inhibition following Revaccination. Change in Fold Inhibition was calculated by subtracting the post-revaccination Fold value from the pre-revaccination fold value. Paired values from before and after revaccination were analyzed with Two-way ANOVA. Fold Change difference with a P<0.05 (vs the pre-immune) was treated as significant

Discussion

Vaccinating Antigen and Highly Responding Cows

Comparison was made in the response induced by different single clade vaccination regimes. AD8, MW, PSC89 as well as KNHISOS-IP strains of gp140 Env were injected into a group of 32 cows over the course of a year. While AD8 had already proven to be a strong immunogen, KNHISOS-IP was thought to be a strong contender due to its stronger mimicry of natural Env. PSC89 was used for being a founder strain isolated from early transmission (before antibody-Env coevolution begins) strains of HIV, thus representing more “contagious” strains of Env. It was found that cows vaccinated with AD8 Env, even those vaccinated with a smaller dose, were more likely to have a higher average OD or titer compared to vaccination with the other antigen strains. Furthermore, vaccination with AD8 raised the broadest response as titers from AD8 samples showed high binding against non-autologous Envelopes, even after losing the “home advantage”. It is also worth noting that the link between binding breadth and binding titer held true on an individual scale as well as on a group level, possibly suggesting binding breadth and binding strength are linked. It is conceivable that strongly binding antibodies bind closer to conserved region of Env, thus increasing cross-clade binding.

These data also suggested that AD8 Env is a superior vaccine antigen relative to the other Env used. One implication of this is that the lower response of tri-mix vaccinations in previous studies could simply be because the non-AD8 Env used in the mixes were less immunogenic and held back the response. This may imply that a tri-mix made of Env strains as equally immunogenic as AD8 could potentially be a superb vaccine.

Antibody Level in Serum

These studies also analyzed the concentration of serum IgG to account for the possibility of the pregnancy confounding results. It was decided to quantify serum IgG and if necessary normalize serum samples via dilution prior to assaying.

It was found that none of the cows had significant increases in serum IgG by the end of the revaccination study, suggesting serum IgG had already recovered within a week of calving. Sample IgG concentrations were relatively consistent between animals and resembled natural variation between dairy cows.

It was decided that samples would not be diluted for later assays. The major reason being that serum IgG concentration reflected the number of HIV specific B-cells within animals, something which would become relevant in future studies with the aim of isolating individual Env-specific B-cells to study the genetics behind the immune response. Diluting samples would risk misrepresenting the relative number of B-cell, making FACs isolation riskier. The other reason for withholding from normalizing serum IgG was that variation in serum IgG may not have necessarily represented a change in anti-HIV envelope antibodies. Beyond natural species variation, it is also possible that IgG levels were higher in some animals due to antibodies being raised against other pathogens since the vaccination study was not held in sterile conditions.

Effectiveness of the SOS-IP Revaccination

Samples of BG505 SOS-IP were obtained and used to produce AD8 SOS-IP.

It was proposed that the different epitopes presented by the cleaved SOS-IP Env may boost the cow's antibodies to greater heights.

These results showed that the AD8 SOS-IP & BG505 SOS-IP revaccinations were able to increase the binding titer of cows who were revaccinated. 5 of the 6 SOS-IP revaccinated animals that were tested showed increases in serum titer vs at least 1 of the tested Env. Curiously, some non SOS-IP control cows also showed increases in titer, which contradicted previous findings showing binding titer had plateaued after a single cycle of vaccination with uncleaved trimer. It is possible that the A-clade epitopes presented by BG505 SEKS were enough to boost the serum response. Another possibility is that anti-HIV maturation was slower and had yet to plateau in those cows, a plausibility since antibody response maturation is a random process with wide temporal variation. Ideally, cows vaccinated with BG505 SEKS for 2 years followed by BG505 SOS-IP could clear these points of contention.

It was noted that BG505 and AD8 SOS-IP Env were able to improve binding titers while KNH1 SOS-IP did not induce a strong response in the initial vaccination. A possible explanation for this is that SOS-IP Env are less immunogenic due to their more compact form, and “priming” with uncleaved Env vaccination is required to jump start the immune system towards making anti-SOS-IP antibodies.

Epitope Targeting of the Response

This study investigated the amount of b12 competition to track how binding against the CD4bs neutralising epitope of Env evolved before and after SOS-IP revaccination. Initial results were consistent with previously published work, as it was also showed CD4bs antibodies had been raised in AD8 Env vaccinated cows within a year of vaccination. Later results were not what was initially expected however, as it was found SOS-IP revaccination did not have a significant impact on CD4bs competition, since competition titer remained stable for the majority (6 out of 7) of samples. Furthermore, the only cows which showed an increase in b12 competition were cows revaccinated with either uncleaved AD8 or AD8 SOS-IP, suggesting b12 competition was raised by AD8 strain epitopes independently of SOS-IP. These plateaued CD4bs titers were in direct contrast to the changes in binding titer, which showed increases for the same samples. Increasing binding titer without a change in CD4bs competition could be explained by the antibody response beginning to target other epitopes.

Finally, it is understood that various other modifications and/or alterations may be made without departing from the spirit of the present invention as outlined herein.

Future patent applications may be filed on the basis of or claiming priority from the present application. It is to be understood that the following claims are provided by way of example only, and are not intended to limit the scope of what may be claimed in any such future application. Features may be added to or omitted from the provisional claims at a later date so as to further define or redefine the invention or inventions.

What is claimed is:

1. A topical formulation for inhibiting transmission of HIV comprising polyclonal antibodies or fragments thereof capable of binding to a human immunodeficiency virus-1 (HIV-1) or human immunodeficiency virus-2 (HIV-2), comprising polyclonal antibodies or fragments thereof capable of binding to a CD4 binding site of a heterologous clade of HIV-1 or HIV-2 viral envelope (Env) protein or a fragment thereof, and
a pharmaceutically acceptable excipient,
wherein the polyclonal antibodies or fragments thereof are produced by immunization of an ungulate animal with a HIV-1 or HIV-2 gp140 (Env) protein or fragment thereof that are obtained from a hyperimmune colostrum of the ungulate animal, or a hyperimmune milk of the ungulate animal, and wherein the polyclonal antibodies are at least partially purified or enriched compared with the hyperimmune colostrum or the hyperimmune milk from which they are obtained.

2. The topical formulation according to claim 1 wherein the gp140 Env protein or fragment thereof is gp140.

3. The topical formulation according to claim 1 wherein the gp140 Env protein or fragment thereof is a gp140 oligomer

4. The topical formulation according to claim 2 wherein the gp140 Env protein or fragment thereof is a gp140 oligomer

5. The topical formulation according to claim 1 wherein the gp140 Env protein or fragment thereof is a stabilized gp140 trimer.

6. The topical formulation according to claim 5 wherein the gp140 trimer is stabilized by way of covalent bond between residues of any two or more of the gp140 trimer.

7. The topical formulation according to claim 6 wherein the covalent bond is formed between a residue of gp120 and a residue of gp41.

8. The topical formulation according to claim 6 wherein the covalent bond is an intermolecular disulphide bond formed between gp120 and gp41.

9. The topical formulation according to claim 8 wherein the stabilized gp140 trimer comprises one or more mutations in gp41 and/or gp120 configured to enhance stability of the trimer.

10. The topical formulation according to claim 9 wherein the mutation is a substitution of a residue in the N-terminal heptad repeat region of gp41.

11. The topical formulation according to claim 10 wherein the mutation is an isoleucine-to-proline substitution at position 559 in the N-terminal heptad repeat region of gp41.

12. The topical formulation according to claim 11, wherein the gp140 Env protein is SOSIP gp140, or functional equivalent thereof.

13. The topical formulation according to claim 1, wherein the ungulate is of the family Bovidae.

14. The topical formulation according to claim 13 wherein the ungulate is a cow.

15. The topical formulation according to claim 1 wherein the polyclonal antibodies or fragments thereof have a subset of antibodies with HCDR3 regions of at least 25 amino acids long.

16. The topical formulation according to claim 1 wherein the polyclonal antibodies or fragments thereof have a subset of antibodies with HCDR3 regions of at least 50 amino acids long.

17. A method for preventing the transmission of HIV from a first person to a second person, comprising applying an effective amount of the topical formulation according to claim 1 to at least one of the first person and the second person.

18. The topical formulation according to claim 1, further comprising a lubricant or an antiviral agent.
